(19) United States
(12) Patent Application Publication
(10) Pub. No.: US 2025/0032710 A1
(43) Pub. Date: Jan. 30, 2025

(54) TIMING AND DOSING IMPROVEMENTS FOR DIABETES MANAGEMENT

(71) Applicants: Dexcom, Inc., San Diego, CA (US); UNIVERSITÀ DEGLI STUDI DI PADOVA, Padova (IT)

(72) Inventors: Jacopo PAVAN, Padova (IT); Giulia NOARO, Padova (IT); Andrea FACCHINETTI, Padova (IT); Giovanni SPARACINO, Padova (IT); Simone DEL FAVERO, Padova (IT); Domenico SALVAGNIN, Padova (IT)

(21) Appl. No.: 18/783,288

(22) Filed: Jul. 24, 2024

Related U.S. Application Data
(60) Provisional application No. 63/515,459, filed on Jul. 25, 2023.

(51) Int. Cl. A61M 5/172; G16H 20/17; G16H 40/67

(52) U.S. Cl. CPC A61M 5/1723; G16H 20/17; G16H 40/67; A61M 2230/201

(57) ABSTRACT

In an embodiment, a system for of preventively treating hypoglycemia includes a continuous glucose monitoring (CGM) sensor system configured to generate measurements associated with a current glucose level of a patient. The system further includes one or more memories comprising executable instructions and one or more processors in data communication with the one or more memories. The one or more processors are configured to execute the executable instructions to receive, from the CGM sensor system, one or more measurements associated with the current glucose level of the patient and compute a sequence of preventive hypoglycemia treatments over a future time period based on the one or more measurements and a prediction of glucose control to be produced by the sequence. The one or more processors are further configured to prompt the patient with a first preventive hypoglycemia treatment in the sequence of preventive hypoglycemia treatments.

### CROSS-REFERENCE TO RELATED APPLICATIONS

This application claims priority to and benefit of U.S. Provisional Patent Application No. 63/515,459, filed Jul. 25, 2023, which is hereby expressly incorporated by reference herein in its entirety as if fully set forth below and for all applicable purposes.

### INTRODUCTION

Type 1 Diabetes (TID) is a chronic autoimmune disease causing insulin deficiency. The lack of insulin results in hyperglycemia, a condition characterized by high blood glucose levels (e.g., glucose levels>180 mg/dl). Hyperglycemia, if persistent, is often responsible for complications to nerves, heart, kidneys and eyes. To avoid these complications, people with TID generally aim to maintain blood glucose concentration within a glycemic safe range known as euglycemia (e.g., blood glucose∈[70,180] mg/dl) by compensating for the lack of insulin exogenously.

Excessive insulin administration can lead to hypoglycemia, a condition characterized by low blood glucose levels (e.g., blood glucose<70 mg/dl). Hypoglycemia is a major obstacle to glycemic control for many patients. If untreated, hypoglycemia can lead to severe complications such as fainting, weakness, coma, or even death. For this reason, glucose levels are typically monitored throughout the day to check whether they fall outside of the glycemic safe range. The monitoring can be accomplished, for example, by self-monitoring blood glucose via fingerstick sampling or by employing a continuous glucose monitoring (CGM) sensor.

### SUMMARY

In an embodiment, a system for preventively treating hypoglycemia includes a continuous glucose monitoring (CGM) sensor system configured to generate measurements associated with a current glucose level of a patient. The system further includes one or more memories comprising executable instructions and one or more processors in data communication with the one or more memories. The one or more processors are configured to execute the executable instructions to receive, from the CGM sensor system, a first one or more measurements associated with the current glucose level of the patient and compute a first sequence of preventive hypoglycemia treatments over a first future time period based on the first one or more measurements and a prediction of glucose control to be produced by the first sequence. The one or more processors are further configured to prompt the patient with a first preventive hypoglycemia treatment in the first sequence of preventive hypoglycemia treatments.

In an embodiment, a method of preventively treating hypoglycemia includes, at user device, receiving, from a continuous glucose monitoring (CGM) sensor system, a first one or more measurements associated with a current glucose level of a patient. The method further includes computing, at the user device, a first sequence of preventive hypoglycemia treatments over a first future time period based on the first one or more measurements and a prediction of glucose control to be produced by the first sequence. The method further includes, at the user device, prompting the patient with a first preventive hypoglycemia treatment in the first sequence of preventive hypoglycemia treatments.

In an embodiment, a computer-program product includes a non-transitory computer-usable medium having computer-readable program code embodied therein. The computer-readable program code is adapted to be executed to implement a method of preventively treating hypoglycemia. The method includes receiving, from a continuous glucose monitoring (CGM) sensor system, a first one or more measurements associated with a current glucose level of a patient. The method further includes computing a first sequence of preventive hypoglycemia treatments over a first future time period based on the first one or more measurements and a prediction of glucose control to be produced by the first sequence. The method further includes prompting the patient with a first preventive hypoglycemia treatment in the first sequence of preventive hypoglycemia treatments.

### BRIEF DESCRIPTION OF THE DRAWINGS

FIG. 1A illustrates an example of a therapy management system, in accordance with certain embodiments.

FIG. 1B illustrates an example analyte sensor system including an example continuous analyte sensor(s) with sensor electronics, in accordance with certain embodiments.

FIG. 2 illustrates example inputs and example outputs that are generated based on the inputs, in accordance with certain embodiments.

FIG. 3 illustrates an example of a process for preventively treating hypoglycemia, in accordance with certain embodiments.

FIG. 4 is a line graph representing glucose concentration levels and alternative hypoglycemia treatments, in accordance with certain embodiments.

FIG. 5 illustrates an example of a penalty function, in accordance with certain embodiments.

FIG. 6 illustrates an example distribution of time below a targeted range for five hypoglycemia treatment methods, in accordance with certain embodiments.

FIG. 7 illustrates hypoglycemic events for five hypoglycemia treatment methods, in accordance with certain embodiments.

FIG. 8 illustrates example treatments for five hypoglycemia treatment methods, in accordance with certain embodiments.

FIG. 9 illustrates a probability of selecting particular treatments for three hypoglycemia treatment methods.

FIG. 10 is a block diagram depicting a computer system configured for facilitating preventive treatment of hypoglycemia.

### DETAILED DESCRIPTION

### 1. Introduction

A major issue with Type 1 diabetes (TID) is the management of hypoglycemia, a condition in which blood glucose levels are low (e.g., glucose levels<70 mg/dl). Low blood glucose levels may cause symptoms in a TID patient such as dizziness, confusion, sweating, weakness, and, in severe cases, loss of consciousness or seizures. These effects may largely be alleviated by administering hypoglycemia treatments or hypotreatments (HTs). HTs can include, for example, consumption of fast-acting carbohydrates (CHOs), injection or infusion of glucagon, and/or taking other actions designed to increase blood glucose levels. For illustrative purposes, examples of HTs are described herein with respect to administering CHOs, though it should be appreciated that the principles described herein are not so limited. Taking CHOs as a running example throughout this Disclosure, in general, the dosage and timing of fast-acting CHOs should be chosen carefully to mitigate a given hypoglycemic event while avoiding large rebounds, which may lead to hyperglycemia.

The American Diabetes Association (ADA) has developed guidelines for the treatment of hypoglycemia, which include consuming any form of fast-acting CHOs containing about 15-20 grams of glucose whenever the TID patient is conscious and blood glucose levels drop below 70 mg/dl. According to the ADA guidelines, the TID patient should re-check their blood glucose levels 15 minutes later and repeat another dose of fast-acting CHOs, if necessary. Following ADA guidelines can potentially reduce the duration and magnitude of hypoglycemic events, but avoiding them in the first place would be preferable so as to minimize the nuisance to the TID patient. For example, following existing guidelines may cause the TID patient to have to frequently re-dose with fast-acting CHOs. Frequent re-dosing may annoy the TID patient, causing the TID patient to ignore the guidelines and potentially risk becoming hypoglycemic.

Accordingly, aspects of the present disclosure provide techniques for improving the management of hypoglycemia through the use of fast-acting CHOs. For example, aspects of the present disclosure provide techniques for predicting future hypoglycemic events and recommending a dosage and timing for consuming fast-acting CHOs to avoid these predicted future hypoglycemic events. Further, the techniques presented herein may reduce the frequency of dosing of the fast-acting CHOs, thereby reducing nuisance to TID patients. Additionally, the techniques presented herein can help quantize suggested dosages to standard, off-the-shelf dosages, thereby avoiding difficulties in determining exactly how much fast-acting CHOs to consume and making it easier for the TID patient to manage their diabetes.

In various embodiments, CHO recommendations may be optimized in the same way as insulin is administered. For example, an amount of administered CHO may vary based on different circumstances.

In various embodiments, one or more model predictive control (MPC) algorithms may be utilized to determine appropriate HTs (e.g., CHO dosing). For example, given a current glucose trace, a model may be implemented (e.g., using one or more MPC algorithms) to determine what happens when various amounts of CHO are administered by a user at different times. See for example, at least Section 3.1 below. The model may be used to determine times and/or amounts that provide desirable results (e.g., by avoiding hypoglycemic and/or hyperglycemic states). A model that avoids a hypoglycemic state while also avoiding a hyperglycemic rebound may be determined to be the most stable or desirable model.

In various embodiments, predetermined guidelines and/or thresholds for dosing may be considered. See for example, at least Equations (1) (quantization) and Equations (2)-(6) (e.g., recommended dosages c_{sugg} (k) and timing between dosages (Δ_{HT})) discussed below. A minimum time between suggested doses may be determined so that the patient is not bothered too frequently to ingest CHO. For example, a matrix representation may be used to implement dosing times guidelines. In another example, a sliding window algorithm may be used with associated constraints (e.g., where only one Boolean value may be implemented within a single window, etc.). Also, only predetermined doses, a predetermined number of doses, etc. may be allowed. For instance, CHO levels may be limited to predetermined amounts (e.g., predetermined doses such as 5 g, 10 g, 15 g, 20 g, etc.). This may be implemented via mutually exclusive Boolean variables, etc.

In various embodiments, a cost function may be implemented to provide CHO recommendations. See, for example, Equations (2) and (3) discussed below. In one example, a cost penalty may be implemented that corresponds to how much a patient's blood glucose deviates from a predetermined standard blood glucose (e.g., a glycemic reference g₀), with the goal of obtaining a stable blood glucose curve. In another example, a cost penalty may be implemented for an increase in a number of CHO doses (e.g., a number of HTs suggestions), with the goal of minimizing a number of CHO doses. In yet another example, a cost penalty may be implemented for an increased amount of CHO intake per dose (with the goal of minimizing an amount of CHO intake per dose). In various embodiments, different cost functions could be considered. For instance, in certain embodiments, an applicable cost function could penalize the amount of suggested CHO instead, or penalize glucose values outside of a target region using a zone penalty.

In various embodiments, one or more predictive models (e.g., machine learning implementations such as neural networks, etc.) may be implemented to provide CHO recommendations.

In various embodiment, parameter tuning may be performed (e.g., to set and adjust a level of aggressiveness, etc.). Optimal desired dosing amounts may be determined utilizing one or more running predictions for one or more patients. For example, one or more patient characteristics (such as body weight) may be input and used to construct a baseline. Various baselines may be created utilizing historical information for a predetermined group of patients. User-input parameters may be used to match a user to a most relevant historical baseline, an optimal rate of CHO ingestion (high q vs low q), etc.

The present disclosure describes an example algorithm for suggesting the dosing and timing of HTs. In various embodiments, the algorithm can address various issues in the prevention and treatment of hypoglycemia. For example, one issue concerns the frequency of CHO intake suggestions. In general, in certain embodiments described herein, HTs are made as infrequent as possible, so as to minimize the nuisance for the patient. In all likelihood, the more frequently the algorithm suggests HTs, the less the patient will comply with these suggestions. Therefore, in certain embodiments, lessening the frequency of HTs improves the patient's time in a glycemic safe range (e.g., blood glucose∈[70,180] mg/dl)).

In certain embodiments, another issue addressed by the algorithm can concern the quantization of the suggestions. In real life, patients cannot be expected to consume any arbitrary amount of CHO suggested by the algorithm. Vice versa, in general, it would be advantageous for the suggestions to be compatible with the common dosages for fast-acting CHO (e.g., dosages in commercially available sugar tabs, or integer multiples of these quantities). Sparsity in time and quantization can both be applied a posteriori (e.g., by rounding the suggested CHO amount to the closest quantization value), but this choice would imply a degradation of the algorithm performances. To overcome this issue, the present disclosure describes examples of integrating these requirements in an optimization procedure by formulating the problem as a constrained integer program (CIP).

Advantageously, in certain embodiments, a decision support system (DSS) implementing the example algorithm described herein can be solely dedicated to HT suggestions without consideration for insulin delivery (e.g., such that the DSS has no authority on insulin delivery). In these embodiments, such a DSS can be compatible with any insulin delivery regimen. In certain embodiments, the algorithm can be easily adapted to insulin regimens and be integrated to the therapy favored by the patient. For example, in various embodiments, the algorithm can be easily adapted to smart insulin pens.

### 2. Example of Computing a Sequence of Preventive HTs

In various embodiments, to intelligently plan future suggestions of HTs, the example algorithm described herein explores possible sequences of future HTs and determines a sequence that minimizes a suitable cost function. In certain embodiments, such a cost function measures an efficacy of the sequence by accounting for a glucose control that the HTs are predicted to produce. Then, following the receding horizon principle, only the first HT of the computed sequence is suggested to the patient and the optimal sequence is updated once a new measurement arrives.

Generalizing the idea of [18], HT suggestions will be denoted with the variable c(k)∈Γ(mg/min), where Γ={0, γ₁, . . . , γ_{NHT}} is the finite set containing the NHT possible dosages of fast-acting CHO that the algorithm can propose.

The variable c(k) can be built as:

$$c ( k ) = {\sum }_{n = 1}^{{N}_{HT}} {γ}_{n} {c}_{n} ( k ) \qquad 1 )$$

where c₁(k), . . . , c_{NHT}(k)∈{0, 1} are mutually exclusive binary variables, i.e. such that Σᵢ₌₁^{N}^{_{HT}}cᵢ(k)≤1. For example, in some embodiments, each binary variable can indicate if the corresponding amount of CHO is selected at instant k: c(k)=γᵢ if and only if cᵢ(k)=1 and cⱼ(k)=0 for all i≠j. This formulation can enforce quantization of the HT suggested and the integer variables introduced can enable less frequent HT suggestions.

In an example, the example algorithm can compute the sequence of HTs [c(k), c(k+1), . . . , c(k+N−1)] that minimizes the cost function:

$$J = {\sum }_{j = 0}^{N - 1} ( {q ( g ( k + j ) - {g}_{0} ( k + j ) )}^{2} + {‖ c ( k ) ‖}_{0} ) \qquad 2 )$$

with N being a finite prediction horizon (PH), set to N=18 sampling instants (corresponding to 1 hour and 30 minutes).

The first term in Equation (2) penalizes the distance between the predicted glucose levels, g(k)∈R (mg/dl), produced by the sequence and a euglycemic setting, such as a euglycemic setpoint, g₀∈R (mg/dl), set to g₀=115 mg/dl in the present example. In some embodiments, the euglycemic setting can correspond to a target range, such as a glycemic safe range as described previously. In these embodiments, the first term in Equation (2) can penalize the distance between the predicted glucose levels and one or more of the target range, a lower boundary of the target range, an upper boundary of the target range, a middle of the target range, and/or the like.

The second term in Equation (2) penalizes the [symbol] ₀ “norm” of c(k),

$${‖ c ( k ) ‖}_{0} = \{ \begin{matrix}0 & if c ( k ) = 0 \\ 1 & if c ( k ) \neq 1\end{matrix} \qquad 2.1 )$$

and thus penalizes the number of (non-zero) HT suggestions in the PH. According to this example, with this choice, only the number of predicted patient interventions is penalized and the amount of CHO suggested is not considered. In certain aspects, this favors larger but rarer CHO suggestions with respect to the standard choice of penalizing the [symbol] ₂ norm of c(k), ∥c(k)∥₂=(c(k))², that instead promotes smaller (but possibly more frequent) HTs.

Finally, it should be noted that

$${‖ c ( k ) ‖}_{0} = {\sum }_{n = 1}^{{N}_{HT}} {‖ {c}_{n} ( k + j ) ‖}_{0} = {\sum }_{n = 1}^{{N}_{HT}} {‖ {c}_{n} ( k + j ) ‖}_{2}^{2} \qquad 3 )$$

meaning that computation of the [symbol] ₀ “norm” of c(k) can be reconducted to a quadratic problem with respect to the variables c₁(k), . . . , c_{NHT}(k), thanks to their binary nature.

The parameter q∈ [symbol] in Equation (2) sets the trade-off between the two penalty terms. The algorithm will often refer to q as “aggressiveness”. A patient-specific tuning of the aggressiveness will be described in Section 4.

The predicted values of g in Equation (2) can be computed, for example, via forward simulation using a linearized physiological model of glucose-insulin dynamics and starting from an estimate of the system state at the current sampling time. The state estimate can be obtained by means of a Kalman Filter leveraging the same model and using the available information on CGM measurements, insulin administration and CHO intake. For sake of disclosure readability, an example of the physiological model is described in Section 3.1, whereas an example of tuning the Kalman Filter is described in Section 3.2.

To enforce a minimal distance in time Δ_{HT} between two consecutive HT, the example algorithm described herein can impose a set of linear inequality constraints on the binary variables c₁(k), . . . , c_{NHT}(k). Focusing on the generic i-th Boolean variable, introduced here are the single Boolean vector Cᵢ(k)=[cᵢ(k), . . . , cᵢ(k+N−1)]^{T} ∈{0, 1}^{N} and the following linear inequality constraint:

$${i}_{} ( k ) \leq f , \\ where \\ = [ {\overbrace{\begin{matrix}1 & … & 1 \\ 0 & 1 & … \\ ⋮ & & ⋱ \\ ⋮ & & \\ 0 & … & …\end{matrix}}}^{{Δ}_{HT}} {\overbrace{\begin{matrix}0 & … & … & 0 \\ 1 & 0 & … & 0 \\ & ⋱ & & ⋮ \\ ⋱ & & ⋱ & 0 \\ 0 & 1 & … & 1\end{matrix}}}^{N - {Δ}_{HT}} ] ∈ , \qquad 4.1 )$$

$$f = [ \begin{matrix}1 \\ ⋮ \\ 1\end{matrix} ] ∈ . \qquad 4.2 )$$

The first row of the inequality in Equation (4), can be rewritten as

$${\sum }_{j = 1}^{{Δ}_{HT}} {c}_{i} ( k + j - 1 ) \leq 1 \qquad 4.3 )$$

Since the elements of cᵢ(k) are Boolean, at most one cᵢ (k+j−1) in the interval j∈[1, Δ_{HT}] can be equal to 1. The second inequality poses a similar constraint on the interval j∈[2, Δ_{HT}+1]. According to this example, the only possible combination of two nonzero actions satisfying both constraints is the one that has cᵢ(k)=1 and cᵢ (k+Δ_{HT}+1)=1. This consideration holds for the entire PH, so that two elements of Cᵢ can be equal to 1 only if they are at least Δ_{HT} elements apart. This kind of constraint can be applied to all the binary variables at the same time:

$${\sum }_{h = j}^{j + {Δ}_{HT} - 1} ( {\sum }_{i = 1}^{{N}_{HT}} {c}_{i} ( k + h ) ) \leq 1 , j = 1 , ... , N - {Δ}_{HT} + 1 \qquad 4.4 )$$

thus preventing to plan two HTs (regardless their amount) at less than Δ_{HT} instants apart. It should be noted that, according to this example, these constraints also automatically impose mutual exclusivity of the binary variables at each time k, thus ensuring that only HT amounts in Γ={γ₁, . . . , γ_{NHT}} are suggested while the sum of different dosages (e.g., γ₁+γ₂) are forbidden in this example.

According to this example, after manipulations known as “condensing,” which are briefly reviewed in Section 3.3, the minimization of the cost function in Equation (2), subject to the binary constraints on c₁, . . . , c_{N}_{_{HT}} and by the sets of affine inequality constraints defined as in Equation (4), the following CIP results:

$${opt}_{} ( k ) = {arg min}_{\begin{matrix}𝒞 ( k ) s . t . ( k ) \leq f \\ 𝒞 ( k ) ∈ {\{ 0 , 1 \}}^{N \cdot {N}_{HT}}\end{matrix}} \frac{1}{2} {T}^{} H + {h}^{T} \qquad 5 )$$

$$where$$

$$( k ) = [ \begin{matrix}{c}_{1} ( k ) \\ ⋮ \\ {c}_{1} ( k + N - 1 ) \\ ⋮ \\ {c}_{{N}_{HT}} ( k ) \\ ⋮ \\ {c}_{{N}_{HT}} ( k + N - 1 )\end{matrix} ] ∈ \qquad 6 )$$

$$= [ \vert ... \vert ] ∈$$

and H and h are suitable matrices derived by straightforwardly representing Equation (2) in matrix notation, see Section 3.3.

The CIP in Equation (5) is solved at each sampling time and from the optimal C found, Cₒₚₜ, the first HT suggestion is derived as follows:

$${c}_{sugg} ( k ) = [ {γ}_{1} , 0 , … , 0 , \vert {γ}_{2} , 0 , … , 0 , \vert … \vert {γ}_{{N}_{HT}} , 0 , … , 0 ] {C}_{opt} . \qquad 7 )$$

If a non-zero HT is suggested at the current instant k and the user confirms the consumption, the algorithm is then shut-off for the following Δ_{HT} sampling instants in order to guarantee the sparsity in time of the suggestions.

Beside suggesting an HT when the first element of the optimal HT sequence is different from zero, the algorithm can also pre-alert the user in case a HT is envisioned in other positions of the PH. In certain embodiments, since the HT strategy in the PH is continuously updated, the planned HT can also be cancelled and the pre-alert withdrawn.

In example periodically described herein, four possible amounts for HT suggestions can be considered (N_{HT}=4): 5, 10, 15 and 20 g. It should be appreciated, however, that the principles described herein are not limited to the foregoing amounts or number of amounts. For purposes of the example periodically described herein, the idea is to suggest integer multiples of a sugar tablet containing, for example, 5 g of CHO. Nevertheless, the framework presented in this Section is easily adaptable to any desired combination of CHO intake. In the example results reported herein, the CIP in Equation (5) is solved by using Matlab and ILOG CPLEX Optimization Studio (v. 12.9) [33].

### 3. Example Prediction Models

### 3.1. Glucose-Insulin Model

In certain embodiments, the example algorithm described herein relies on the prediction of future glycemic levels. To this purpose, in certain embodiments, the algorithm can employ, for example, an averaged, linearized and discretized version of the physiological model of glucose-insulin dynamics within a TID simulator (see, e.g., [36]). In state space form, the model can be represented as:

$$\{ \begin{matrix}\overline{x} ( k + 1 ) = A \overline{x} ( k ) + {B}_{i} \overline{ι} ( k ) + {B}_{m} \hat{m} ( k ) + {B}_{c} c ( k ) \\ \overline{g} ( k ) = C \overline{x} ( k )\end{matrix} \qquad 8 )$$

The state vector, x̅(k)∈ [symbol] , is the deviation of the state of the system x(k) from the operating point around which the model was linearized, x_{eq}. The output variable, g̅(k)∈ [symbol] , (mg/dl), is the deviation of glucose levels g(k) from a patient-specific equilibrium point G_{b}, called basal glucose. The variable ι̅(k)∈ [symbol] (pmol/kg/min) is the normalized insulin administration, defined as

$$\overline{ι} ( k ) = \frac{i ( k ) - {i}_{b} ( k )}{BW} \qquad 9 )$$

where i(k) ∈ [symbol] (pmol/min) is the total insulin administration, i_{b}(k)∈ [symbol] (pmol/min) is patient's basal insulin administration and BW (Kg) is patient's body weight. The difference i(k)−i_{b}(k) accounts for all insulin administration exceeding basal needs, e.g., prandial or corrective boluses.

The variable \hat{m}(k)∈ [symbol] (mg/min) represent the announced meal intake, i.e., the CHO content of the meal as estimated by patients. This variable could be affected by counting errors.

The variable c(k) represents HT suggestions. By expressing c(k) as the linear combination of mutually exclusive binary variables, it is constrained to only assume values in I. At difference with ι̅(k) and \hat{m}(k), c(k) can be manipulated by the example algorithm described herein to mitigate hypoglycemic events. Advantageously, in certain embodiments, the model represented by Equation (8) is both stabilizable and detectable.

### 3.2. Tuning the Kalman Filter

The prediction of future glycemic levels in Equation (2) can be obtained via forward simulation from an estimate of the current system state, \hat{x}(k)∈ [symbol] , and by no meal consumption and basal insulin infusion along the PH (see Section 3.3). For example, this estimate can be obtained, at each sampling instant, by relying on a Kalman Filter. Following [49], the measurement noise covariance matrix is set to Σᵥ=9.8496 mg²/dl². The process noise covariance matrix Σ_{w}∈R^{13×13} is a diagonal matrix whose diagonal elements w₁, . . . , w₁₃ are reported in Table I.

TABLE I

| Elements | Value [%] | Measurement unit |
|---|---|---|
| w1, w2, w3 | 0.1 | mg² |
| w4, w5 | 10 | mg²/kg² |
| w6, w7, w8 | 0.1 | pmol²/l² |
| w9, w10, w11, w12 | 0.1 | pmol²/kg² |
| w13 | 10 | mg²/dl² |

### 3.3. Condensing

Let k be the current instant. By leveraging the state evolution law described in (8), any future glycemic value g⁻(k+j), with j=1, . . . , N−1, can be iteratively predicted from the current state estimate x\hat{ }(k) as:

$$\overline{g} ( k + j ) = CA \hat{x} ( k + j ) ++ {\sum }_{n = 0}^{j - 1} {CA}^{j - n - 1} ( {B}_{i} \overline{ι} ( k + n ) + {B}_{m} \hat{m} ( k + n ) + {B}_{c} c ( k + n ) ) \qquad 10 )$$

that in matrix notation reads

$$( k ) = \hat{x} ( k ) + {i}_{} ( k ) + {m}_{} + {c}_{} ( k ) \qquad 11 )$$

$$where$$

$$( k ) = [ \begin{matrix}\overline{g} ( k ) \\ ⋮ \\ \overline{g} ( k + N - 1 )\end{matrix} ] , = [ \begin{matrix} \\ ⋮ \\ {N}^{}\end{matrix} ] , \qquad 12 )$$

$${i}_{} = [ \begin{matrix}{i}_{} & 0 & … & 0 \\ {i}_{} & {i}_{} & ⋱ & 0 \\ ⋮ & ⋱ & ⋱ & 0 \\ {N}^{} {i}_{} & {N - 1}^{} {i}_{} & … & \end{matrix} ] , \qquad 13 )$$

$${m}_{} = [ \begin{matrix}{m}_{} & 0 & … & 0 \\ {m}_{} & {m}_{} & ⋱ & 0 \\ ⋮ & ⋱ & ⋱ & 0 \\ {N}^{} {m}_{} & {N - 1}^{} {m}_{} & … & {m}_{}\end{matrix} ] , \qquad 14 )$$

$${c}_{} = [ {γ}_{1} {{\_}^{}}_{c} , … , {γ}_{{N}_{HT}} {{\_}^{}}_{c} ] , \qquad 15 )$$

$${{\_}^{}}_{c} = [ \begin{matrix}{{\_}^{}}_{c} & 0 & … & 0 \\ {{\_}^{}}_{c} & {{\_}^{}}_{c} & ⋱ & 0 \\ ⋮ & ⋱ & ⋱ & 0 \\ {N}^{} {{\_}^{}}_{c} & {N - 1}^{} {{\_}^{}}_{c} & … & {{\_}^{}}_{c}\end{matrix} ] , \qquad 16 )$$

$$ℐ ( k ) = [ \begin{matrix}\overline{ι} ( k ) \\ 0 \\ ⋮ \\ 0\end{matrix} ] , ( k ) = [ \begin{matrix}\hat{m} ( k ) \\ 0 \\ ⋮ \\ 0\end{matrix} ] , \qquad 17 )$$

and C(k) defined as in Equation (6). As insulin boluses and meal intake are relatively rare events during the day, the vectors [symbol] (k) and [symbol] (k) are supposed to be 0 in the PH, with the only exception of insulin boluses administered and meals announced at the current instant k(ι̅(k) and \hat{m}(k), respectively.

The cost function in Equation (2) can then be reformulated in matrix notation as:

$$J = {( - {0}_{} )}^{T} ( - {0}_{} ) + \qquad 18 )$$

$$where$$

$$= [ \begin{matrix}q & 0 & … & 0 \\ 0 & q & & ⋮ \\ ⋮ & & ⋱ & 0 \\ 0 & … & 0 & q\end{matrix} ] , {0}_{} = [ \begin{matrix}{g}_{0} ( k ) \\ ⋮ \\ {g}_{0} ( k + N - 1 )\end{matrix} ] \qquad 19 )$$

By substituting Equation (11) in Equation (18), obtained is:

$${J = ( \hat{x} ( k ) + {i}_{} ( k ) + {m}_{} ( k ) ) ++ {C}_{} ( k ) )}^{T} \text{} \text{} ( + \hat{x} ( k ) + {i}_{} ( k ) ++ {m}_{} ( k ) + {C}_{} ( k ) ) + {( k )}^{T} ( k ) \qquad 20 )$$

This can be expressed in a classical integer quadratic program form as:

$$J = \frac{1}{2} {C ( k )}^{T} HC ( k ) + {h}^{T} C ( k ) \qquad 21 )$$

$$with$$

$$H = {B}_{c}^{T} {QB}_{c} + I \qquad 22 )$$

$$h = {( \hat{x} ( k ) + {i}_{} ( k ) + {m}_{} ( k ) - {0}_{} )}^{T} {C}_{}$$

and I being an identity matrix of appropriate size.

### 4. Aggressiveness Tuning

In certain embodiments, the aggressiveness, i.e., the hyper-parameter q in Equation (2), can play a contributory role: the higher q, the more the algorithm is prone to suggest HTs for getting closer to the reference; the lower q, the more the utilization of CHO is discouraged.

As glucose metabolism largely varies among individuals, it may be hard to find a single value of q that well suits any subject. For this reason, aggressiveness needs to be optimized for each individual.

In general, optimizing q by trial and error would involve testing several different tunings of this parameter on a single subject and to choose the value that achieves the best glycemic regulation. Although such a procedure might be easy to perform in simulation, in certain embodiments, it may be suboptimal in vivo, as testing multiple tunings could be burdensome and potentially dangerous for patients. Therefore, leveraging subject matter described in [33], the algorithm can use the simulator to find an optimal tuning of the aggressiveness and then use these optimal values to train a regression law which aim is to infer a personalized value of q from patients-specific physical or therapeutic parameters, e.g., their body weight.

An example simulation described herein exploited 100 virtual subjects of the TID Simulator, split them in a training and a test set (75 and 25 subjects, respectively), and designed the following tuning pipeline:
1) simulate 3 days of simulation on the training set for several values of q;
2) find the optimal value of q for each subject as the one that minimizes some cost function;
3) train a regression law from the optimal values of q and subjects-specific parameters;
4) evaluate the performances of the algorithm on 3 days of simulation with the suboptimal tuning of q on the subjects of the test set.

### 4.1. Optimal Tuning for Virtual Subjects

The optimal value of q can be searched via grid search in the interval [10⁻⁷, 10⁻²]. This interval was selected based on a preliminary analysis. Forty log-spaced values are tested in this interval. The optimal value of q is chosen as the one that minimizes the following cost function:

$${\sum }_{k = 0}^{{T}_{end}} D ( q , k ) \qquad 23 )$$

$$where$$

$$D ( q , k ) = \{ \begin{matrix}{α ( 70 - g ( q , k ) )}^{2} & if g ( q , k ) < 70 \\ {β ( g ( q , k ) - 180 )}^{2} & if g ( q , k ) > 180 \\ 0 & otherwise\end{matrix} \qquad 24 )$$

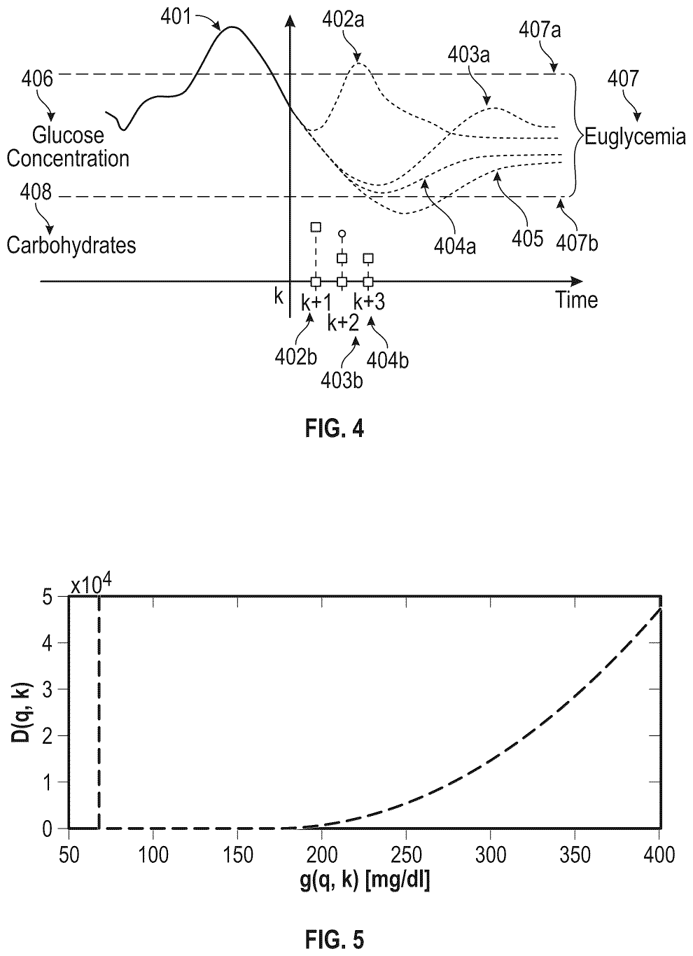

With g(q,k)∈ [symbol] in Equation (24), the output CGM signal obtained with a specific tuning q is indicated. The function in Equation (24) assigns a penalty to each measurement based on the square of its distance from the euglycemic interval. The parameters α and β assign different weights to the penalties for measurements in hypoglycemia and hyperglycemia. The parameters are set at α=104 and β=1 to penalize hypoglycemia more than hyperglycemia. The resulting penalty function D(q,k) is plotted in FIG. 5.

### 4.2. Control Aggressiveness Estimation

Consider a generic training subject p. The optimal control aggressiveness q(p) can be computed for this subject as described in Section 4.1. Here the algorithm derives a regression model to predict the optimal tuning considering different easily accessible and subject-specific parameters as regressors, for example: the insulin-to-carbohydrate ratio (CR(p)) and the correction factor (CF(p)), i.e., two patient-specific therapy parameters that are tuned by the clinician [34], the basal insulin infusion (I_{b}(p)), the basal glucose level (G_{b}(p)) and the body weight (BW(p)) of the subject.

For this purpose, a least absolute shrinkage and selection operator (LASSO) regression model can be employed to tackle the possible correlation among the features, by performing both variable selection and regularization. The resulting model has the following form:

$$\hat{q} ( p ) = 0.014 \cdot CR ( p ) + 0.033 \cdot CF ( p ) + 0.726 \cdot {I}_{b} ( p ) - 0.002 \cdot BW ( p ) + 0.043 \cdot {G}_{b} ( p ) - 11.302 . \qquad 25 )$$

where \hat{q}(p) is the tuning parameter estimated by the model.

The above formula can be used for any new subject (including real subjects), providing an estimate of the optimal tuning, that can be used without requiring to run the possibly dangerous grid search procedure. In addition, or alternatively, in some embodiments, the aggressiveness q (p) can be set by a patient or other user, for example, based on their risk tolerance and/or particularized knowledge of the patient's health.

### 5. Benchmark Algorithms, Experimental Setup and Assessment Criteria

### 5.1. Benchmarks Algorithms

This Section compares the example algorithm described above to various other algorithms described in publications listed herein. In particular, the example algorithm will be compared to the standard ADA guidelines [19], the enhanced ADA strategy proposed in Vettoretti et al. [20], and the method proposed in Camerlingo et al. [13], hereafter labelled as ADA, Enhanced ADA, and Dynamic Risk, respectively. In addition, the example algorithm will be compared to a HT algorithm based a population autoregressive and moving average model with exogenous inputs (ARMAX) model as proposed in [35]. For purposes of this comparison, the example algorithm described herein will be periodically referenced in the Drawings as “the proposed method.”

### 5.2. Simulation Scenario

This example simulation uses the TID simulator described in [36]. This tool is accepted by the Food and Drug Administration as an alternative to animal tests and, since 2008, it has been widely used by the scientific community.

The core of this simulator is a large-scale mathematical model of glucose metabolism in TID subjects, which consists of 13 differential equations and more than 30 parameters.

The model is equipped with a cohort of 100 virtual adult patients, that is, 100 different realizations of the parameters of the model. Each set of parameters is sampled from a joint distribution, which is inferred from unique clinical data [37]; hence, the correlation among parameters respects the one observed in real subjects.

One of the most noticeable features of this latest version of the TID simulator is time variability. It includes a model of the so-called “dawn phenomenon” and intra-day variations of insulin sensitivity that well mimic the daily patterns observed in vivo [38]. The pattern of insulin sensitivity is also subjected to random inter-day variations. CGM sensor measurement error is implemented as in [39].

The performances of the different hypo-treating strategies will be evaluated on 3 days of simulation, with each day characterized by three meals: breakfast, lunch and dinner. Meal timing and CHO content are drawn from uniform distributions to match the data reported in [40]. Table II reports the bounds of these distributions. Several studies reports how counting the correct CHO content of a meal is a challenging task for individuals with TID and that carb-counting errors (CCEs) generally correlate with meal CHO amount and type [41]. In order to mimic this aspect, the CHO content that subjects announce to the HT algorithms can be affected by a CCE computed as in [41].

Insulin administration is simulated as in a standard, open-loop regimen for continuous subcutaneous insulin infusion [42], i.e., using an insulin infusion pump. This regimen consists of a basal insulin infusion, administered during the whole day to meet non-prandial insulin needs, and meal-time insulin boli, which is used to cover the meal CHO content. Basal insulin will be simulated with a step-wise constant signal. Insulin boli will be delivered exclusively when a meal is consumed, and the amount of insulin in the bolus will be determined using the so-called standard formula [43]:

$${i}_{bolus} ( k ) = \frac{\hat{m} ( k )}{CR} + \frac{g ( k ) - {g}_{0}}{CF} - 10 B \qquad 26 )$$

The variable \hat{m}(k) indicates the CHO amount of the meal as estimated by patients (i.e., accounting for CCE). The insulin-on-board, IOB, is the quantity of active insulin in the organism, and it is computed using a 6 hours insulin action curve as in [44].

TABLE II

| Meal | CHO amount (g) | Time (daytime) |
|---|---|---|
| breakfast | [19-97] | [6.30 am-8.00 am] |
| lunch | [31-124] | [11.30 am-1.00 pm] |
| dinner | [28-140] | [6.00 pm-8.30 pm] |

To guarantee a fair comparison of different HT strategies on the same subject, each virtual subject will show the same realization of the random factors by fixing individual seeds in the generator of random numbers.

### 5.3. Assessment Criteria

In order to carry out an unbiased evaluation of the example algorithm described herein, the comparison with the benchmark methods was performed on the test subjects only, i.e. on the randomly extracted virtual subjects that were not used for the regression of the optimal q. The simulation scenario lasted 3 days and included by the variability sources explained in Section 5.2.

The test evaluated the glycemic control by computing four metrics commonly used for such a purpose, namely the percentage of time spent within the target glycemic range (TIR [%]), i.e., [70, 180] mg/dl, above 180 mg/dl, and below 70 mg/dl (TAR [%], and TBR [%] respectively), together with the number of hypoglycemic events [46], and the number of subjects experiencing hypoglycemia. In addition, to quantify the burden in terms of therapeutic actions introduced by the example algorithm compared to the benchmark, the simulation computed the average number and grams of hypo-treatments suggested per day.

To assess statistical differences in the glycemic metrics and amount/number of HTs between the example algorithm described herein and the benchmark methods, the simulation involved a paired test for metrics having Gaussian distribution, and a Wilcoxon signed rank test for metrics that have not. Gaussianity was determined via Lilliefors test, with a significance level of 5%. Statistical differences between the number of subjects experiencing hypoglycemia are tested using a Fisher exact test. In all these tests, the simulation corrected the significance level of 5% for multiple comparisons via Bonferroni method and used a significance level of 1.25%.

### 5.4. Results

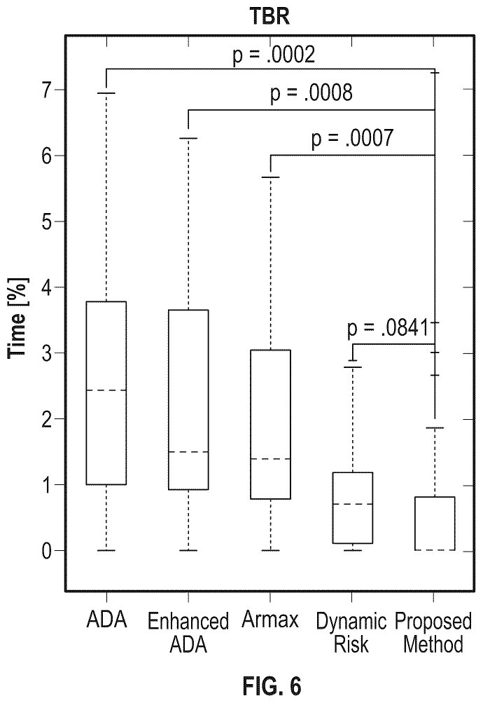

Table III reports the median and interquartile range of TBR, TIR, and TAR metrics on the test patients, while FIG. 6 shows the distribution of TBR for the five hypo-treating methods, with the corresponding p-value on the test patients. As can be noted from both Table III and FIG. 6, the example algorithm described herein outperforms the benchmarks when considering the TBR, achieving a median of 0%, together with the lowest 75th percentile. Indeed, the highest median is reached by the standard ADA guidelines (2.43%), followed by the enhanced ADA (1.50%), the ARMAX model (1.39%), and lastly the dynamic risk algorithm (0.69%). In addition, the methodology described herein resulted in statistically significant improvement in terms of TBR, as compared to standard ADA, enhanced ADA, and ARMAX.

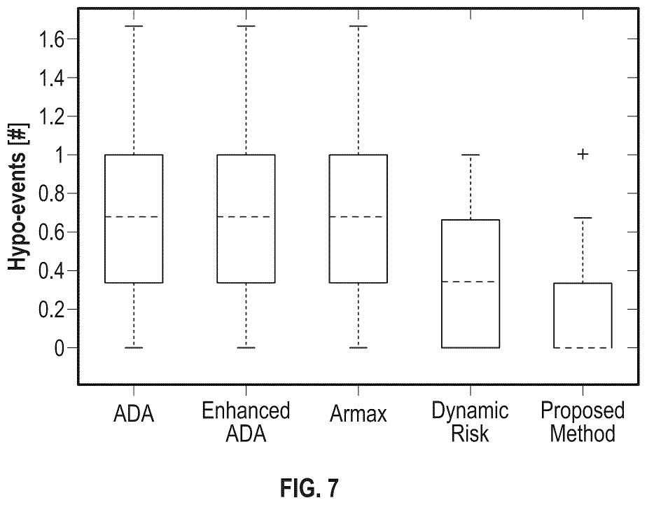

The positive results concerning the TBR are also confirmed by the number of hypoglycemic events that occurred on average during one day for each hypo-treating strategy, as shown in FIG. 7. The use of the algorithm has considerably reduced the onset of hypoglycemia, leading to a median of 0 events per day, as opposed to the other methods, which show a median of 0.7 events for standard ADA, enhanced ADA, and ARMAX, and 0.3 for dynamic risk method. The reduction in the number of hypoglycemic events is statistically significant with respect to ADA, Enhanced ADA and ARMAX (p-value<0.001). In addition, as shown in Table III, the TIR achieved by the ADA guidelines is inferior, with respect the example algorithm described herein (p-value=0.008).

Of the 25 subjects in the test set, 23 experienced hypoglycemia when treated with ADA guidelines, enhanced ADA guidelines and with the algorithm based on ARMAX prediction. This number decreases to 20 with the algorithm based on dynamic risk and to just 11 subjects when using the example algorithm described herein. A Fisher exact test confirmed a non-random association between the number of subjects experiencing hypoglycemia with the first three methods versus the example algorithm described herein (p-value<0.001)

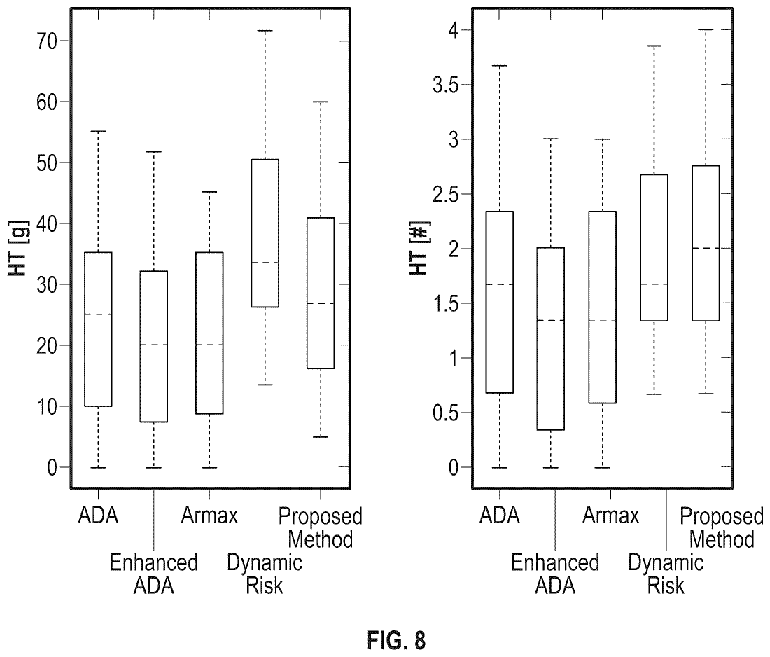

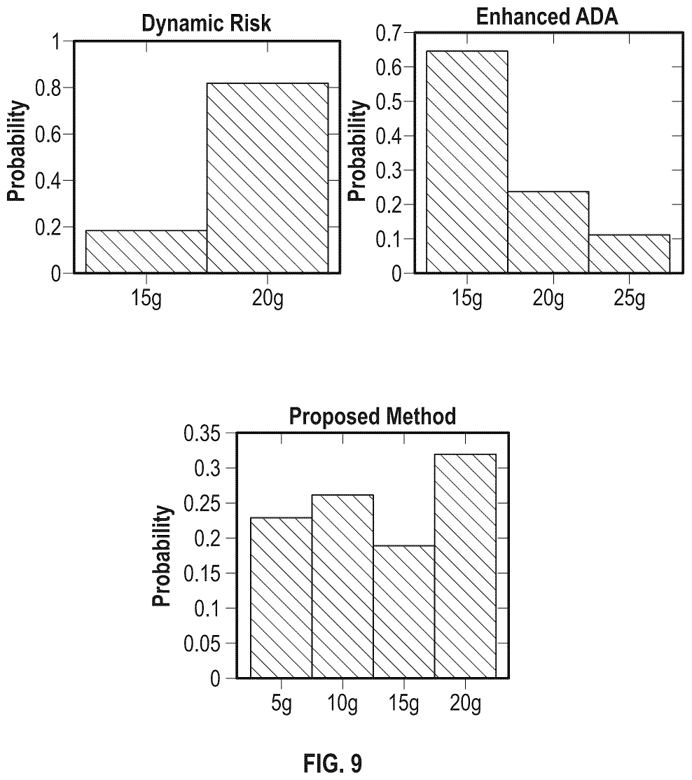

Moreover, the burden introduced by the use of the example algorithm described herein was evaluated with respect to the benchmarks, in terms of amount (FIG. 8, left panel) and number (FIG. 8, right panel) of HT suggested on average during one day. The frequency of selected HT amounts was analyzed for methods that use different possible HT quantities (e.g., enhanced ADA, dynamic risk, and the example algorithm described herein) referred to in FIG. 9. This figure shows that the example algorithm reduced the HT gram amount compared to the dynamic risk algorithm, and did not result in a major increase of HT gram amount compared to the standard ADA, enhanced ADA, and ARMAX models. Moreover, while the majority of dynamic risk and enhanced ADA suggested HT is equal to 20 and 15 grams respectively, the example algorithm described herein proves to evenly use all the available quantities.

TABLE III

| | | | | | Proposed |
|---|---|---|---|---|---|
| | ADA guidelines | Enhanced ADA | ARMAX prediction | Dynamic risk | method |
| TBR [%] | 2.43 | 1.50 | 1.39 | 0.69 | 0.00 |
| median [25^{th}-75^{th} | (1.01, 3.79) | (0.93, 3.65) | (0.78, 3.04) | (0.12, 1.19) | (0.00, 0.81) |
| percentile] | | | | | |
| TIR [%] | 70.49 (±9.62) | 71.11 (±9.86) | 71.68 (±9.75) | 71.36 (±7.93) | 72.45 (±10.27) |
| mean (±std | | | | | |
| deviation) | | | | | |
| TAR [%] | 27.54 | 27.56 | 27.77 | 28.23 | 28.70 |
| median [25^{th}-75^{th} | (21.60, 33.14) | (21.03, 33.32) | (20.89, 23.21) | (23.22, 32.34) | (21.14, 31.38) |
| percentile] | | | | | |

### 6. Example Implementation in a Therapy Management System

In certain embodiments, the example algorithm described above can be implemented in a therapy management system operable to perform continuous analyte monitoring. As used herein, the term “continuous” analyte monitoring refers to monitoring one or more analytes in a fully continuous, semi-continuous, periodic manner, which results in a data stream of analyte values over time. A data stream of analyte values over time is what allows for meaningful data and insights to be derived using the algorithms described herein for preventively treating hypoglycemia. In other words, single point-in-time measurements collected as a result of a patient visiting their health care professional every few months results in sporadic data points (e.g., that are, at best, months apart in timing) that cannot form the basis of any meaningful data or insight to be derived. As such, without the continuous analyte monitoring system of the embodiments herein, it is simply impossible to continuously suggest preventive HTs, as described herein.

Further, the data stream of analyte values collected over time, with the continuous analyte monitoring system presented herein, include real-time analyte values, which allows for deriving meaningful data and insight in real-time using the systems and algorithms described herein. The derived real-time data and insight in turn allows for providing real-time HT suggestions to prevent hypoglycemia. Real-time analyte values herein refer to analyte values that become available and actionable within seconds or minutes of being produced as a result of at least one sensor electronics module of the continuous analyte monitoring system (1) converting sensor current(s) (i.e., analog electrical signals) generated by the continuous analyte sensor(s) into sensor count values, (2) calibrating the count values to generate at least glucose and/or other analyte concentration values using calibration techniques described herein to account for the sensitivity of the continuous analyte sensor(s), and (3) transmitting measured glucose and/or other analyte concentration data, including glucose and/or other analyte concentration values, to a display device via wireless connection.

For example, the at least one sensor electronics module may be configured to sample the analog electrical signals at a particular sampling period (or rate), such as every 1 second (1 Hz), 5 seconds, 10 seconds, 30 seconds, 1 minute, 3 minutes, 5 minutes, etc., and to transmit the measured glucose and/or other analyte concentration data to a display device at a particular transmission period (or rate), which may be the same as (or longer than) the sampling period, such as every 1 minute (0.016 Hz), 5 minutes, 10 minutes, etc.

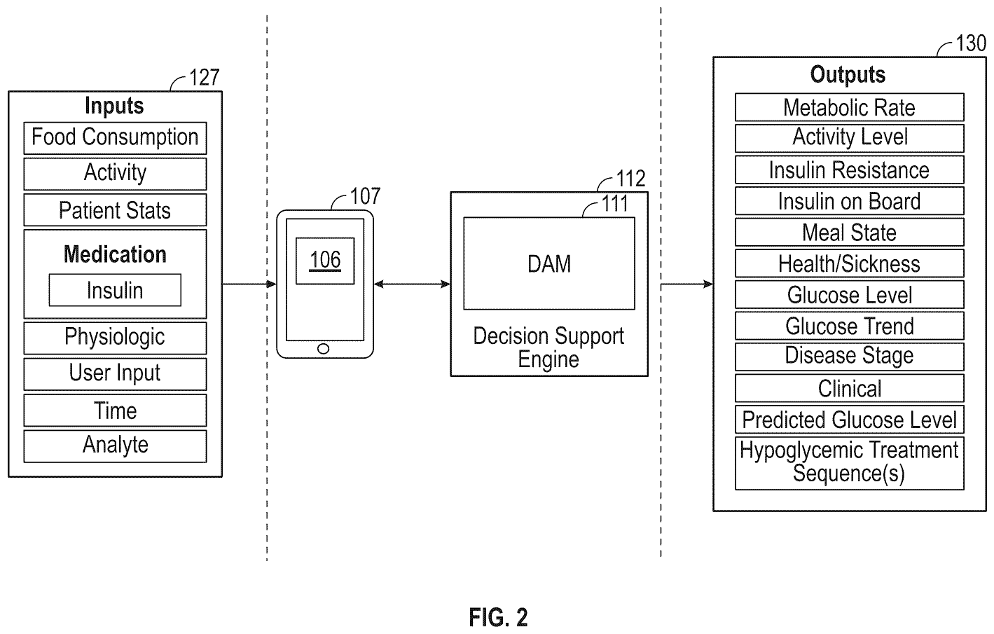

The real-time analyte data that is continuously generated by the continuous analyte monitoring system described herein, therefore, allows the therapy management system herein to suggest preventive HTs, in real-time, which is technically impossible to perform using existing or conventional techniques or systems. Further, because of the real-time nature of this data, it is also humanly impossible to continuously process a real-time data stream of analyte values over time to derive meaningful data and insight using the algorithms and systems described herein to generate and suggest preventive HTs to a patient. In other words, deriving meaningful data and insight from a stream of real-time data that is continuously generated, processed, calibrated, and analyzed, using the algorithms and systems described herein, is not a task that can be mentally performed. For example, executing the algorithms described above in Sections 1-5 and/or below in relation to FIG. 3, in real-time and on a continuous basis, which would involve using a stream of real-time data that is continuously generated by a patient's continuous analyte monitoring system and/or significantly large amount of population data (e.g., hundreds or thousands of data points for each one of thousands or millions of patients in the patient population) is not a task that can be mentally performed, especially in real-time at times.

Further, certain embodiments herein are directed to a technical solution to a technical problem associated with analyte sensor systems. In particular, each analyte sensor system that is manufactured by a sensor manufacturer might perform slightly differently. As such, there might be inconsistencies between sensors and the measurements they generate once in use. Accordingly, certain embodiments herein are directed to determining the performance of an analyte sensor system during a manufacturing calibration process (in vitro), which includes quantifying certain sensor operating parameters, such as a calibration slope (also known as calibration sensitivity), a calibration baseline, etc.

Generally, calibration sensitivity refers to the amount of electrical current produced by an analyte sensor of an analyte sensor system when immersed in a predetermined amount of a measured analyte. The amount of electrical current may be expressed in units of picoAmps (pA) or counts. The amount of measured analyte may be expressed as a concentration level in units of milligrams per deciliter (mg/dL), and the calibration sensitivity may be expressed in units of pA/(mg/dL) or counts/(mg/dL). The calibration baseline refers to the amount of electrical current produced by the analyte sensor when no analyte is detected, and may be expressed in units of pA or counts.

The calibration sensitivity, calibration baseline, and other information related to the sensitivity profile for the analyte sensor system may be programmed into the sensor electronics module of the analyte sensor system during the manufacturing process, and then used to convert the analyte sensor electrical signals into measured analyte concentration levels. For example, the calibration slope (calibration sensitivity) may be used to predict an initial in vivo sensitivity (M₀) and a final in vivo sensitivity (M_{f}), which are programmed into the sensor electronics module and used to convert the analyte sensor electrical signals into measured analyte concentration levels.

In certain embodiments, during in vivo use, the sensor electronics module of an analyte sensor system samples the analog electrical signals produced by the analyte sensor to generate analyte sensor count values, and then determines the measured analyte concentration levels based on the analyte sensor count values, the initial in vivo sensitivity (M₀), and the final in vivo sensitivity (M_{f}). For example, measured analyte concentration levels may be determined using a sensitivity function M(t) that is based on the initial in vivo sensitivity (M₀) and the final in vivo sensitivity (M_{f}). The sensitivity function M(t) may expressed in several different ways, such as a simple correction factor that is not dependent on elapsed time (tᵢ) of in vivo use, a linear relationship between sensitivity and time (tᵢ), an exponential relationship between sensitivity and time (tᵢ), etc. Equation 27 presents one technique for determining a measured analyte concentration level (ACL) from an analyte sensor count value (count) at a time tᵢ:

$$ACL = count / M ( {t}_{i} ) \qquad 27 )$$

A calibration baseline (baseline) may also be used to determine a measured analyte concentration level (ACL) from an analyte sensor count value (count) at a time tᵢ, and Equation 28 presents one technique:

$$ACL = ( count - baseline ) / M ( {t}_{i} ) \qquad 28 )$$

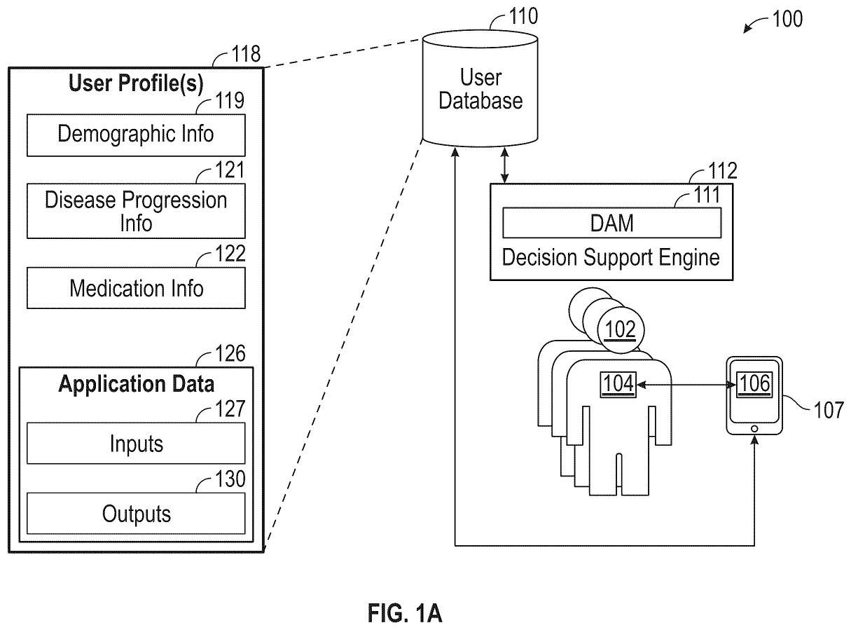

FIG. 1A illustrates an example of a therapy management system 100 implementing the example algorithm discussed above in Sections 1 to 5, in accordance with certain embodiments of the disclosure. The therapy management system 100 may be utilized for generating and presenting information related to user health, for example, using various user interfaces associated with system 100. Each user of system 100, such as user 102, may interact with a mobile health application, such as mobile health application (“application”) 106 (e.g., a diabetes intervention application that provides therapy management guidance), and/or a health monitoring device, such as an analyte sensor system 104 (e.g., a glucose monitoring system). User 102, in certain embodiments, may be the patient or, in some cases, the patient's caregiver. In the embodiments described herein, the user is assumed to be the patient for simplicity only, but is not so limited. As shown, system 100 may include an analyte sensor system 104, a display device 107 that executes application 106, a decision support engine 112, and a user database 110.

Analyte sensor system 104 may be configured to generate time-series data, such as analyte measurements (e.g., sensor data), for the user 102, e.g., on a continuous basis, and transmit the analyte measurements to the display device 107 for use by application 106. In some embodiments, the analyte sensor system 104 may transmit the analyte measurements to the display device 107 through a wireless connection (e.g., Bluetooth connection). In certain embodiments, display device 107 is a smart phone. However, in certain embodiments, display device 107 may instead be any other type of computing device such as a laptop computer, a smartwatch, a tablet, or any other computing device capable of executing application 106.

Note that, while in certain examples the analyte sensor system 104 is assumed to be a glucose monitoring system, analyte sensor system 104 may operate to monitor one or more additional or alternative analytes. As discussed, the term “analyte” as used herein is a broad term, and is to be given its ordinary and customary meaning to a person of ordinary skill in the art (and is not to be limited to a special or customized meaning), and refers without limitation to a substance or chemical constituent in the body or a biological sample (e.g., bodily fluids, including, blood, serum, plasma, interstitial fluid, cerebral spinal fluid, lymph fluid, ocular fluid, saliva, oral fluid, urine, excretions, or exudates).

Analytes can include naturally occurring substances, artificial substances, metabolites, and/or reaction products. In some embodiments, the analyte measured and used by the devices and methods described herein may include albumin, alkaline phosphatase, alanine transaminase, aspartate aminotransferase, bilirubin, blood urea nitrogen, calcium, CO2, chloride, creatinine, glucose, gamma-glutamyl transpeptidase, hematocrit, lactate, lactate dehydrogenase, magnesium, oxygen, pH, phosphorus, potassium, ketones, sodium, total protein, uric acid, metabolic markers, and/or drugs.

Other analytes are contemplated as well, including but not limited to acetaminophen, dopamine, ephedrine, terbutaline, ascorbate, uric acid, oxygen, d-amino acid oxidase, plasma amine oxidase, xanthine oxidase, NADPH oxidase, alcohol oxidase, alcohol dehydrogenase, pyruvate dehydrogenase, diols, Ros, NO, bilirubin, cholesterol, triglycerides, gentisic acid, ibuprophen, L-Dopa, methyl dopa, salicylates, tetracycline, tolazamide, tolbutamide, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments.

The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), histamine, Advanced Glycation End Products (AGEs) and 5-hydroxyindoleacetic acid (FHIAA).

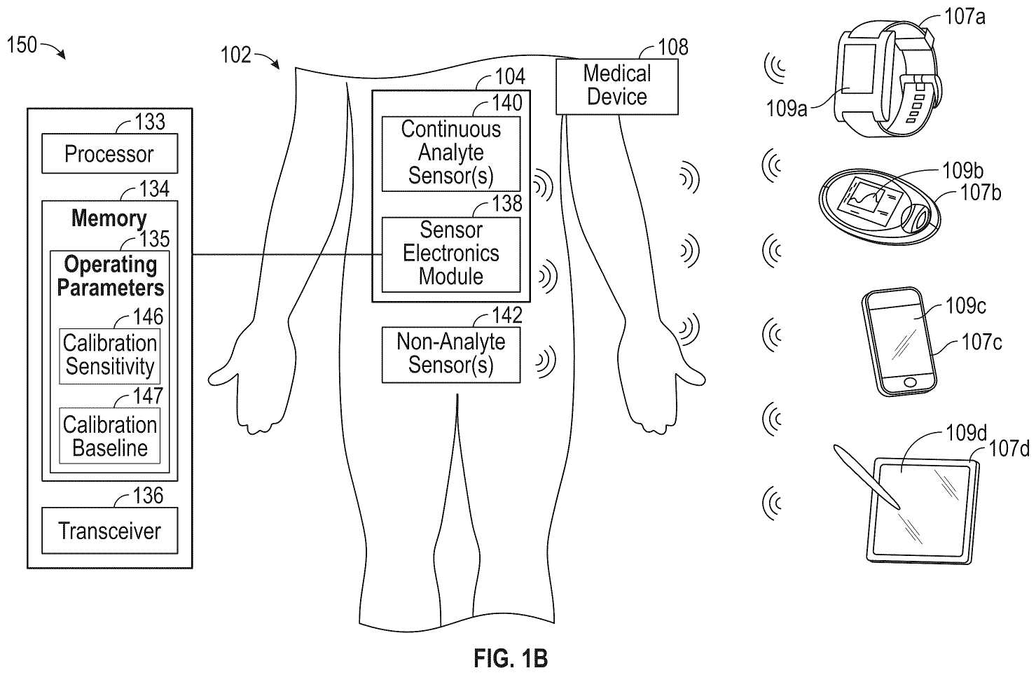

Application 106 may be a mobile health application that is configured to receive and analyze time-series data, including analyte measurements, from the analyte sensor system 104 and/or other devices, as described in greater detail relative to FIGS. 1B and 2. In some embodiments, application 106 may transmit analyte measurements received from the analyte sensor system 104 to a user database 110 (and/or the decision support engine 112), and the user database 110 (and/or the decision support engine 112) may store the analyte measurements in a user profile 118 of user 102 for processing and analysis, for example, by the decision support engine 112. In some embodiments, application 106 may store the analyte measurements in a user profile 118 of user 102 locally for processing and analysis, for example, by the decision support engine 112.

In certain embodiments, application 106 is configured to provide various interfaces for receiving, from the user 102, data of the type discussed previously. In an example, the application 106 can provide a user interface that enables the user 102 to graphically respond to the decision support engine 112 to confirm HTs (e.g., suggestions of CHO dosages) from the decision support engine 112 for user 102, and/or that enables the application 106 to perform other functions such as indicating a CHO dosage.

In certain embodiments, decision support engine 112 refers to a set of software instructions with one or more software modules, including a data analysis module (DAM) 111. In some embodiments, decision support engine 112 executes entirely on one or more computing devices in a private or a public cloud. In some other embodiments, decision support engine 112 executes partially on one or more local devices, such as display device 107 (e.g., via application 106) and/or analyte sensor system 104, and partially on one or more computing devices in a private or a public cloud. In some other embodiments, decision support engine 112 executes entirely on one or more local devices, such as display device 107 (e.g., via application 106) and/or analyte sensor system 104. In certain embodiments, decision support engine 112, via the application 106, may provide interfaces for suggesting and confirming recommended CHO dosages.

In certain embodiments, DAM 111 of decision support engine 112 may be configured to receive and/or process a set of inputs 127 (described in more detail below) (also referred to herein as “input data”) to determine one or more outputs 130 (also referred to herein as “metrics data”). Inputs 127 may be stored in the user profile 118 in the user database 110. DAM 111 can fetch inputs 127 from the user database 110 and compute a plurality of outputs 130 which can then be stored as application data 126 in the user profile 118. Such outputs 130 may include health-related metrics.

In certain embodiments, application 106 is configured to take as input information relating to user 102 and store the information in a user profile 118 for user 102 in user database 110. For example, application 106 may obtain and record user 102's demographic info 119, disease progression info 121, and/or medication info 122 in user profile 118. In certain embodiments, demographic info 119 may include one or more of the user's age, body mass index (BMI), ethnicity, gender, etc. In certain embodiments, disease progression info 121 may include information about the user 102's disease, such as, for diabetes, whether the user is Type I, Type II, pre-diabetes, or whether the user has gestational diabetes. In certain embodiments, disease progression info 121 also includes the length of time since diagnosis, the level of disease control, level of compliance with disease management therapy, predicted pancreatic function, other types of diagnosis (e.g., heart disease, obesity) or measures of health (e.g., heart rate, exercise, stress, sleep, etc.), and/or the like. In certain embodiments, medication info 122 may include information about the amount and type of medication taken by user 102, such as insulin or non-insulin diabetes medications and/or non-diabetes medication taken by user 102.

In certain embodiments, application 106 may obtain demographic info 119, disease progression info 121, and/or medication info 122 from the user 102 in the form of user input or from other sources. In certain embodiments, as some of this information changes, application 106 may receive updates from the user 102 or from other sources. In certain embodiments, user profile 118 associated with the user 102, as well as other user profiles associated with other users are stored in a user database 110, which is accessible to application 106, as well as to the decision support engine 112, over one or more networks (not shown).

In certain embodiments, application 106 collects inputs 127 through user 102 input and/or a plurality of other sources, including analyte sensor system 104, other applications running on display device 107, and/or one or more other sensors and devices. In certain embodiments, such sensors and devices include one or more of, but are not limited to, an insulin pump, other types of analyte sensors, sensors or devices provided by display device 107 (e.g., accelerometer, camera, global positioning system (GPS), heart rate monitor, etc.) or other user accessories (e.g., a smartwatch), or any other sensors or devices that provide relevant information about the user 102. In certain embodiments, user profile 118 also stores application configuration information indicating the current configuration of application 106, including its features and settings.

User database 110, in some embodiments, refers to a storage server that may operate in a public or private cloud. User database 110 may be implemented as any type of data store, such as relational databases, non-relational databases, key-value data stores, file systems including hierarchical file systems, and the like. In some exemplary implementations, user database 110 is distributed. For example, user database 110 may comprise a plurality of persistent storage devices, which are distributed. Furthermore, user database 110 may be replicated so that the storage devices are geographically dispersed.

User database 110 may include other user profiles 118 associated with a plurality of other users served by therapy management system 100. More particularly, similar to the operations performed with respect to the user 102, the operations performed with respect to these other users may utilize an analyte monitoring system, such as analyte sensor system 104, and also interact with the same application 106, copies of which execute on the respective display devices of the other users 102. For such users, user profiles 118 are similarly created and stored in user database 110.

FIG. 1B is a diagram 150 conceptually illustrating an example continuous analyte monitoring system 104 including example continuous analyte sensor(s) with sensor electronics, in accordance with certain aspects of the present disclosure. For example, system 104 may be configured to continuously monitor one or more analytes of a patient, in accordance with certain aspects of the present disclosure.

Continuous analyte monitoring system 104 in the illustrated embodiment includes sensor electronics module 138 and one or more continuous analyte sensor(s) 140 (individually referred to herein as continuous analyte sensor 140 and collectively referred to herein as continuous analyte sensors 140) associated with sensor electronics module 138. Sensor electronics module 138 may be in wireless communication (e.g., directly or indirectly) with one or more of display devices 107a, 107b, 107c, and 107d. In certain embodiments, sensor electronics module 138 may also be in wireless communication (e.g., directly or indirectly) with one or more medical devices, such as medical devices 108 (individually referred to herein as medical device 108 and collectively referred to herein as medical devices 108), and/or one or more other non-analyte sensors 142 (individually referred to herein as non-analyte sensor 142 and collectively referred to herein as non-analyte sensor 142).

In certain embodiments, a continuous analyte sensor 140 may comprise one or more sensors for detecting and/or measuring analyte(s). The continuous analyte sensor 140 may be a multi-analyte sensor configured to continuously measure two or more analytes or a single analyte sensor configured to continuously measure a single analyte as a non-invasive device, a subcutaneous device, a transcutaneous device, a transdermal device, and/or an intravascular device. In certain embodiments, the continuous analyte sensor 140 may be configured to continuously measure analyte levels of a patient using one or more techniques, such as enzymatic techniques, chemical techniques, physical techniques, electrochemical techniques, spectrophotometric techniques, polarimetric techniques, calorimetric techniques, iontophoretic techniques, radiometric techniques, immunochemical techniques, and the like. The term “continuous,” as used herein, can mean fully continuous, semi-continuous, periodic, etc. In certain aspects, the continuous analyte sensor 140 provides a data stream indicative of the concentration of one or more analytes in the patient. The data stream may include raw data signals, which are then converted into a calibrated and/or filtered data stream used to provide estimated analyte value(s) to the patient.

In certain embodiments, the continuous analyte sensor 140 may be a multi-analyte sensor, configured to continuously measure multiple analytes in a patient's body. For example, in certain embodiments, the continuous multi-analyte sensor 140 may be a single sensor configured to measure lactate, glucose, ketones (e.g., 3-beta-hydroxybutyrate, acetoacetate, acetone, etc.), glycerol, and/or free fatty acids in the patient's body.

In certain embodiments, one or more multi-analyte sensors may be used in combination with one or more single analyte sensors. As an illustrative example, a multi-analyte sensor may be configured to continuously measure lactate and glucose and may, in some cases, be used in combination with an analyte sensor configured to measure only ketones or only potassium. Information from each of the multi-analyte sensor(s) and single analyte sensor(s) may be combined to provide therapy management support using methods described herein. In further embodiments, other non-contact and or periodic or semi-continuous, but temporally limited, measurements for physiological information may be integrated into the system such as by including weight scale information or non-contact heart rate monitoring from a sensor pad under the patient while in a chair or bed, through an infra-red camera detecting temperature and/or blood flow patterns of the patient, and/or through a visual camera with machine vision for height, weight, or other parameter estimation without physical contact.

In certain embodiments, the continuous analyte sensor(s) 140 may comprise a percutaneous wire that has a proximal portion coupled to the sensor electronics module 138 and a distal portion with several electrodes, such as a measurement electrode and a reference electrode. The measurement (or working) electrode may be coated, covered, treated, embedded, etc., with one or more chemical molecules that react with a particular analyte, and the reference electrode may provide a reference electrical voltage. The measurement electrode may generate the analog electrical signal, which is conveyed along a conductor that extends from the measurement electrode to the proximal portion of the percutaneous wire that is coupled to the sensor electronics module 138. After the continuous analyte monitoring system 104 has been applied to epidermis of the patient, continuous analyte sensor(s) 140 penetrates the epidermis, and the distal portion extends into the dermis and/or subcutaneous tissue under epidermis. Other configurations of continuous analyte sensor(s) 140 may also be used, such as a multi-analyte sensor that includes multiple measurement electrodes, each generating an analog electrical signal that represents the concentration levels of a particular analyte.

Generally, a single-analyte sensor generates an analog electrical signal that is proportional to the concentration level of a particular analyte. Similarly, each multi-analyte sensor generates multiple analog electrical signals, and each analog electrical signal is proportional to the concentration level of a particular analyte. As an illustrative example, continuous analyte sensor 140 may include a single-analyte sensor configured to measure lactate concentration levels, and another single-analyte sensor configured to measure glucose concentration levels of the patient. As another illustrative example, continuous analyte sensor(s) 140 may include a single-analyte sensor configured to measure glucose concentration levels, and one or more multi-analyte sensors configured to measure lactate concentration levels, ketone concentration levels, creatinine concentration levels, etc. As yet another illustrative example, continuous analyte sensor(s) 140 may include a multi-analyte sensor configured to measure lactate concentration levels, glucose concentration levels, ketone concentration levels, creatinine concentration levels, etc. Accordingly, continuous analyte sensor(s) 140 is configured to generate at least one analog electrical signal that is proportional to the concentration level of a particular analyte, and sensor electronics module 138 is configured to convert the analog electrical signal into an analyte sensor count values, calibrate the analyte sensor count values based on the sensitivity profile of the continuous analyte sensor(s) 140 to generate measured analyte concentration levels, and transmit the measured analyte concentration level data, including the measured analyte concentration levels, to a display device, such as display devices 107b, 107c, and/or 107d, via a wireless connection. For example, sensor electronics module 138 may be configured to sample the analog electrical signal at a particular sampling period (or rate), such as every 1 second (1 Hz), 5 seconds, 10 seconds, 30 seconds, 1 minute, 3 minutes, 5 minutes, etc., and to transmit the measured analyte concentration data to the display device at a particular transmission period (or rate), which may be the same as (or longer than) the sampling period, such as every 1 minute (0.016 Hz), 5 minutes, 10 minutes, 30 minutes, at the conclusion of the wear period, etc. Depending on the sampling and transmission periods, the measured analyte concentration data transmitted to the display device include at least one measured analyte concentration level having an associated time tag, sequence number, etc.

In certain embodiments, continuous analyte sensor(s) 140 may incorporate a thermocouple within, or alongside, the percutaneous wire to provide an analog temperature signal to the sensor electronics module 138, which may be used to correct the analog electrical signal or the measured analyte data for temperature. In other embodiments, the thermocouple may be incorporated into the sensor electronics module 138 above the adhesive pad, or, alternatively, the thermocouple may contact the epidermis of the patient through openings in the adhesive pad.

In certain embodiments, the sensor electronics module 138 includes, inter alia, processor 133, storage element or memory 134, wireless transmitter/receiver (transceiver) 136, one or more antennas coupled to wireless transceiver 136, analog electrical signal processing circuitry, analog to-digital (A/D) signal processing circuitry, digital signal processing circuitry, a power source for continuous analyte sensor(s) 140 (such as a potentiostat), etc.

Processor 133 may be a general-purpose or application-specific microprocessor, an application-specific integrated circuit (ASIC), a field programmable gate array (FPGA), etc., that executes instructions to perform control, computation, input/output, etc, functions for the sensor electronics module 138. Processor 133 may include a single integrated circuit, such as a micro processing device, or multiple integrated circuit devices and/or circuit boards working in cooperation to accomplish the appropriate functionality. In certain embodiments, processor 133, memory 134, wireless transceiver 136, the A/D signal processing circuitry, and the digital signal processing circuitry may be combined into a system-on-chip (SoC).

Generally, processor 133 may be configured to sample the analog electrical signal using the A/D signal processing circuitry at regular intervals (such as the sampling instant or period) to generate analyte sensor count values based on the analog electrical signals produced by the continuous analyte sensor(s) 140, calibrate the analyte sensor count values based on the sensitivity profile of the continuous analyte sensor(s) 140 to generate measured analyte concentration levels, and generate measured analyte data from the measured analyte concentration levels, generate sensor data packages that include, inter alia, the measured analyte concentration level data. Processor 133 may store the measured analyte concentration level data in memory 134, and generate the sensor data packages at regular intervals (such as the transmission period) for transmission by wireless transceiver 136 to a display device, such as display devices 107b, 107c, 107d, and/or 107a. Processor 133 may also add additional data to the sensor data packages, such as supplemental sensor information that includes a sensor identifier, a sensor status, temperatures that correspond to the measured analyte data, etc. The sensor data packages are then wirelessly transmitted over a wireless connection to the display device. In certain embodiments, the wireless connection is a Bluetooth or Bluetooth Low Energy (BLE) connection. In such embodiments, the sensor data packages are transmitted in the form of Bluetooth or BLE data packets to the display device

In various embodiments, memory 134 may include volatile and nonvolatile medium. For example, memory 134 may include combinations of random access memory (RAM), dynamic RAM (DRAM), static RAM (SRAM), read only memory (ROM), flash memory, cache memory, and/or any other type of non-transitory computer-readable medium. Memory 134 may store one or more analyte sensor system applications, modules, instruction sets, etc. for execution by processor 133, such as instructions to generate measured analyte data from the analyte sensor count values, etc.

Memory 134 may also store certain sensor operating parameters 135, such as a calibration slope (or calibration sensitivity), a calibration baseline, etc. In particular, the calibration sensitivity, calibration baseline, and other information related to the sensitivity profile for the sensor electronics module 138 may be programmed into the sensor electronics module 138 during the manufacturing process, and then used to convert the analyte sensor electrical signals into measured analyte concentration levels. For example, as discussed above, the calibration slope may be used to predict an initial in vivo sensitivity (M₀) and a final in vivo sensitivity (M_{f}), which are stored in memory 134 and used to convert the analyte sensor electrical signals into measured analyte concentration levels. In certain embodiments, calibration sensitivity (M_{CC}) 146 and/or calibration baseline 147 may be stored in memory 134.

In certain embodiments, sensor electronics module 138 includes electronic circuitry associated with measuring and processing the continuous analyte sensor data, including prospective algorithms associated with processing and calibration of the sensor data. Sensor electronics module 138 can be physically connected to continuous analyte sensor(s) 140 and can be integral with (non-releasably attached to) or releasably attachable to continuous analyte sensor(s) 140. Sensor electronics module 138 may include hardware, firmware, and/or software that enable measurement of levels of analyte(s) via continuous analyte sensor(s) 140. For example, sensor electronics module 138 can include a potentiostat, a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor.

Display devices 107b, 107c, 107d, and/or 107a are configured for displaying displayable sensor data, including analyte data, which may be transmitted by sensor electronics module 138. Each of display devices 107b, 107c, 107d, or 107a may include a display such as a touchscreen display 109b, 109c, 109d, and/or 109a for displaying sensor data to a patient and/or for receiving inputs from the patient. For example, a graphical user interface (GUI) may be presented to the patient for such purposes. In certain embodiments, the display devices may include other types of user interfaces such as a voice user interface instead of, or in addition to, a touchscreen display for communicating sensor data to the patient of the display device and/or for receiving patient inputs. Display devices 107a, 107b, 107c, and 107d may be examples of display device 107 illustrated in FIG. 1 used to display sensor data to a patient of the system of FIG. 1 and/or to receive input from the patient.

In certain embodiments, one, some, or all of the display devices are configured to display or otherwise communicate (e.g., verbalize) the sensor data as it is communicated from the sensor electronics module (e.g., in a customized data package that is transmitted to display devices based on their respective preferences), without any additional prospective processing required for calibration and real-time display of the sensor data.

The plurality of display devices may include a custom display device specially designed for displaying certain types of displayable sensor data associated with analyte data received from sensor electronics module. In certain embodiments, the plurality of display devices may be configured for providing alerts/alarms based on the displayable sensor data. Display device 107b is an example of such a custom device. In certain embodiments, one of the plurality of display devices is a smartphone, such as display device 107c which represents a mobile phone, using a commercially available operating system (OS), and configured to display a graphical representation of the continuous sensor data (e.g., including current and historic data). Other display devices can include other hand-held devices, such as display device 107d which represents a tablet, display device 107a which represents a smart watch or fitness tracker, medical device 108 (e.g., an insulin delivery device or a blood glucose meter), and/or a desktop or laptop computer (not shown).

Because different display devices provide different user interfaces, content of the data packages (e.g., amount, format, and/or type of data to be displayed, alarms, and the like) can be customized (e.g., programmed differently by the manufacture and/or by an end user, such as the patient) for each particular display device. Accordingly, in certain embodiments, a plurality of different display devices can be in direct wireless communication with a sensor electronics module (e.g., such as an on-skin sensor electronics module 138 that is physically connected to continuous analyte sensor(s) 140) during a sensor session to enable a plurality of different types and/or levels of display and/or functionality associated with the displayable sensor data.

As mentioned, sensor electronics module 138 may be in communication with a medical device 108. Medical device 108 may be a passive device in some example embodiments of the disclosure. For example, medical device 108 may be an insulin pump for administering insulin to a patient. For a variety of reasons, it may be desirable for such an insulin pump to receive and track lactate, glucose, ketones, glycerol and free fatty acid values transmitted from continuous analyte monitoring systems 104, where continuous analyte sensor 140 is configured to measure lactate, glucose, ketones, glycerol, and/or free fatty acids.

Further, as mentioned, sensor electronics module 138 may also be in communication with other non-analyte sensors 142. Non-analyte sensors 142 may include, but are not limited to, an altimeter sensor, an accelerometer sensor, a global positioning system (GPS) sensor, a temperature sensor, a respiration rate sensor, etc. Non-analyte sensors 142 may also include monitors such as heart rate monitors, blood pressure monitors, pulse oximeters, caloric intake monitors, indirect calorimetry devices, continuous positive airway pressure machines, and medicament delivery devices. One or more of these non-analyte sensors 142 may provide data to decision support engine 112 described further below. In some aspects, a patient may manually provide some of the data for processing by the decision support engine 112 of FIG. 1.

In certain embodiments, non-analyte sensors 142 may further include sensors for measuring skin temperature, core temperature, sweat rate, and/or sweat composition.

In certain embodiments, the non-analyte sensors 142 may be combined in any other configuration, such as, for example, combined with one or more continuous analyte sensors 140. As an illustrative example, a non-analyte sensor, e.g., a temperature sensor, may be combined with a continuous glucose sensor 140 to form a glucose/temperature sensor used to transmit sensor data to the sensor electronics module 138 using common communication circuitry. As another illustrative example, a non-analyte sensor, e.g., a temperature sensor, may be combined with a multi-analyte sensor 140 configured to measure lactate and glucose to form a lactate/glucose/temperature sensor used to transmit sensor data to the sensor electronics module 138 using common communication circuitry.

In certain embodiments, a wireless access point (WAP) may be used to couple one or more of continuous analyte monitoring system 104, the plurality of display devices, medical device(s) 108, and/or non-analyte sensor(s) 142 to one another. For example, such WAP may provide Wi-Fi and/or cellular connectivity among these devices. Near Field Communication (NFC) and or Bluetooth may also be used among devices depicted in diagram 150 of FIG. 1B.

FIG. 2 illustrates example inputs and example metrics that are generated based on the inputs in accordance with certain embodiments of the disclosure. In particular, FIG. 2 illustrates example inputs 127 on the left, application 106 and decision support engine 112, with DAM 111, in the middle, and example outputs 130 on the right. In certain embodiments, application 106 may obtain inputs 127, in the form of time-series data, through one or more channels (e.g., continuous analyte sensor(s) 104, non-analyte sensor(s) 142, various applications executing on display device 107, etc.). Inputs 127 may be further processed by DAM 111 to output a plurality of metrics, such as outputs 130. Further, inputs (e.g., inputs 127) and metrics (e.g., outputs 130) may be used by the DAM 111 and/or any computing device in the system 100 to perform various processes. Any of inputs 127 may be used for computing any of outputs 130. In certain embodiments, each one of outputs 130 may correspond to one or more values, e.g., discrete numerical values, ranges, or qualitative values (high/medium/low or stable/unstable). In some embodiments, some or all of outputs 130 may include time-series data and/or be provided in the form of time-series data.

In certain embodiments, inputs 127 include food consumption information. Food consumption information may include information about one or more of meals, snacks, and/or beverages, such as one or more of the size, content (carbohydrate, fat, protein, etc.), sequence of consumption, and time of consumption. In certain embodiments, food consumption may be provided by the user through manual entry, by providing a photograph through an application that is configured to recognize food types and quantities, and/or by scanning a bar code or menu. In various examples, meal size may be manually entered as one or more of calories, quantity (e.g., ‘three cookies’), menu items (e.g., ‘Royale with Cheese’), and/or food exchanges (1 fruit, 1 dairy). In some examples, meals may also be entered with the user's typical items or combinations for this time or setting (e.g., workday breakfast at home, weekend brunch at restaurant). In some examples, meal information may be received via a convenient user interface provided by application 106.

In certain embodiments, inputs 127 include activity information. Activity information may be provided, for example, the one or more non-analyte sensors 142 of FIG. 1B. In certain embodiments, activity information may additionally be provided through manual input by user 102. Activity information may include, for example, a time series for each of heart rate, activity minutes, step count, floors climbed, location information (e.g., GPS data), calories burned, sleep duration and/or quality, activity level (e.g., light, medium, or heavy), and/or similar information. In addition, or alternatively, the activity information can include one or more time series for recorded activities of one or more defined activity types (e.g., walk, run, sprint, swim, weightlift etc.), where each activity is associated with a duration and/or time period.

In certain embodiments, inputs 127 include patient statistics, such as one or more of age, height, weight, body mass index, body composition (e.g., % body fat), stature, build, or other information. Patient statistics may be provided through a user interface, by interfacing with an electronic source such as an electronic medical record, and/or from measurement devices. The measurement devices may include one or more of a wireless, e.g., Bluetooth-enabled, weight scale and/or camera, which may, for example, communicate with the display device 107 to provide patient data.

In certain embodiments, inputs 127 include information relating to the user's medication intake. For example, the user's medication intake may include the user's insulin delivery. Such information may be received, via a wireless connection on a smart pen, via user input, and/or from an insulin pump (e.g., medical device 108). Insulin delivery information may include one or more of insulin volume, time of delivery, etc. Other configurations, such as insulin action time or duration of insulin action, may also be received as inputs.

In certain embodiments, inputs 127 include physiological information received from non-analyte sensor(s) 142, which may detect one or more of heart rate, respiration, oxygen saturation, body temperature, etc. (e.g., to detect illness, stress levels, etc.). In certain embodiments, inputs 127 include time, such as time of day, or time from a real-time clock.

In certain embodiments, inputs 127 include analyte data, which may be provided as input from analyte sensor system 104, for example, in any of the ways described with respect to FIG. 1A. An example of analyte data is glucose data, which may be provided and/or stored as a time series corresponding to time-stamped glucose measurements over time. Other types of analyte data, such as ketone data, potassium data, lactate data, etc., may similarly be provided and/or stored as a time series.

As described above, in certain embodiments, DAM 111 generates, determines, and/or computes outputs 130 based on inputs 127 associated with user 102. An example list of outputs 130 is illustrated in FIG. 2. In certain embodiments, outputs 130 generated, determined, or computed by DAM 111 include metabolic rate. Metabolic rate is a metric that may indicate or include a basal metabolic rate (e.g., energy consumed at rest) and/or an active metabolism, e.g., energy consumed by activity, such as exercise or exertion. In some examples, basal metabolic rate and active metabolism may be tracked as separate metric. In certain embodiments, the metabolic rate may be calculated by DAM 111 based on one or more of inputs 127, such as one or more of activity information, sensor input, time, user input, etc.

In certain embodiments, outputs 130 generated, determined, or computed by DAM 111 include an activity level metric. The activity level metric may indicate a level of activity of the user. In certain embodiments, the activity level metric may be determined, for example based on input from an activity sensor or other physiologic sensors. In certain embodiments, the activity level metric may be calculated by DAM 111 based on one or more of inputs 127, such as one or more of activity information, physiological information, analyte data, time, user input, etc. Activity level may indicate whether the user is exercising, at rest, sleeping, etc.

In certain embodiments, outputs 130 generated, determined, or computed by DAM 111 include an insulin resistance metric (also referred to herein as an “insulin resistance”). The insulin resistance metric may be determined using historical data, real-time data, or a combination thereof, and may, for example, be based upon one or more inputs 127, such as one or more of food consumption information, blood glucose information, insulin delivery information, the resulting glucose levels, etc. In certain embodiments, the insulin on board metric may be determined using insulin delivery information, and/or known or learned (e.g., from patient data) insulin time action profiles, which may account for both basal metabolic rate (e.g., update of insulin to maintain operation of the body) and insulin usage driven by activity or food consumption.

In certain embodiments, outputs 130 generated, determined, or computed by DAM 111 include a meal state metric. The meal state metric may indicate the state the user is in with respect to food consumption. For example, the meal state may indicate whether the user is in one of a fasting state, pre-meal state, eating state, post-meal response state, or stable state. In certain embodiments, the meal state may also indicate nourishment on board, e.g., meals, snacks, or beverages consumed, and may be determined, for example from food consumption information, time of meal information, and/or digestive rate information, which may be correlated to food type, quantity, and/or sequence (e.g., which food/beverage was eaten first.).

In certain embodiments, outputs 130 generated, determined, or computed by DAM 111 include health and sickness metrics. Health and sickness metrics may be determined, for example, based on one or more of user input (e.g., pregnancy information or known sickness information), from non-analyte sensor(s) 142, such as physiologic sensors (e.g., temperature), activity sensors, or a combination thereof. In certain embodiments, based on the values of the health and sickness metrics, for example, the user's state may be defined as being one or more of healthy, ill, rested, or exhausted. In certain embodiments, health and sickness metric may indicate the user's heart rate, stress level, etc.

In certain embodiments, outputs 130 generated, determined, or computed by DAM 111 include analyte level metrics. Analyte level metrics may be determined from analyte data (e.g., glucose measurements obtained from analyte sensor system 104). In some examples, an analyte level metric may also be determined, for example, based upon historical information about analyte levels in particular situations, e.g., given a combination of food consumption, insulin, and/or activity. An analyte level metric may include a rate of change of the analyte, time in range, time spent below a threshold level, time spent above a threshold level, or the like. In certain embodiments, an analyte trend may be determined based on the analyte level over a certain period of time. As described above, example analytes may include glucose, ketones, lactate, potassium and others described herein.

In certain embodiments, outputs 130 generated, determined, or computed by DAM 111 include a disease stage. For example disease stages for Type II diabetics may include a pre-diabetic stage, an oral treatment stage, and a basal insulin treatment stage. In certain embodiments, degree of glycemic control (not shown) may also be determined as an outcome metric, and may be based, for example, on one or more of glucose levels, variation in glucose level, or insulin dosing patterns.

In certain embodiments, outputs 130 generated, determined, or computed by DAM 111 include clinical metrics. Clinical metrics generally indicate a clinical state a user is in with respect to one or more conditions of the user, such as diabetes. For example, in the case of diabetes, clinical metrics may be determined based on glycemic measurements, including one or more of A1C, trends in A1C, time in range, time spent below a threshold level, time spent above a threshold level, and/or other metrics derived from glucose values. In certain embodiments, clinical metrics may also include one or more of estimated A1C, glycemic variability, hypoglycemia, and/or health indicator (time magnitude out of target zone).

In certain embodiments, outputs 130 generated, determined, or computed by DAM 111 can include predicted glucose levels. For example, in various embodiments, the DAM 111 can compute the predicted glucose levels as discussed above in Section 3.

In certain embodiments, outputs 130 generated, determined, or computed by DAM 111 can include one or more HT sequences. Each HT sequence can include, for example, HT suggestions (e.g., CHO dosages) over a period of time. For example, in various embodiments, the DAM 111 can determine when a patient will need to take a HT as well as suggest present or future HTs. The DAM 11 can compute each HT sequence, for example, as discussed above in Sections 1-5.

It should be appreciated that, although the foregoing examples of outputs 130 are presented for illustrative purposes, the principles described herein are not limited to the presented examples. In various embodiments, outputs 130 can also include, for example, insulin sensitivity, insulin-to-carbohydrate ratio, a correction factor, and/or other data or metrics that may be suitable in a given implementation.

FIG. 3 illustrates an example of a process 300 for preventively treating hypoglycemia using the example algorithm described in Sections 1-5. In some embodiments, the process 300 can be executed, for example, by the decision support engine 112 of FIGS. 1A-B and 2. In addition, or alternatively, the process 300 can be executed, for example, by the application 106 of FIGS. 1A-B and 2. In addition, or alternatively, the process 300 can be executed generally by any of the display devices 107 of FIGS. 1A-B and 2. Although any number of systems, in whole or in part, can implement the process 300, to simplify discussion, the process 300 will be described primarily in relation to the decision support engine 112 of FIGS. 1A-B and 2.

At block 302, the decision support engine 112 receives glucose data that can include, for example, a current glucose level of the patient, as discussed relative to FIGS. 1A-B and 2. At block 304, the decision support engine 112 computes a sequence of preventive HTs for a future time period (e.g., a PH) based on the glucose data and a prediction of glucose control produced by the sequence. The sequence can be computed, for example, as discussed above in Section 2. The block 304 can include, for example, updating or replacing sequences that were computed in any previous iterations of the block 304. More generally, the sequence can modify the sequence that was computed in the most recent iteration of the block 304. At block 306, the decision support engine 112 prompts the patient with the first preventive HT in the sequence of preventive HTs, for example, as an HT suggestion, as discussed above in Section 2.

At decision block 308, the decision support engine 112 determines whether the first preventive HT in the computed sequence has been delivered to (e.g., consumed by) the patient in correspondence to the prompt. For example, as discussed above, in some embodiments, the decision support engine 112 can receive, in an interface of the application 106, a confirmation that the first preventive HT has been delivered (e.g., consumed by) the patient. If a negative determination is reached at the decision block 308, the process 300 returns to the block 302 and executes as described previously. If it is determined, at the decision block 308, that the first preventive HT in the computed sequence has been delivered to (e.g., consumed by) the patient, the process 300 proceeds to block 310.

In some embodiments, decision block 308 can be replaced by, or supplemented with, utilization of an automatic meal detection algorithm. In these embodiments, a meal indicated by the automatic meal detection algorithm can function as confirmation that the first preventive HT in the computed sequence has been delivered to (e.g., consumed by) the patient.

At block 310, in some embodiments, the process 300 can shut down for a predetermined period of time, such as a sampling instant or an integer multiple thereof. Advantageously, in certain embodiments, as discussed above, shutting down the process 300 for the predetermined period of time can increase an amount of time between HTs. In these embodiments, at the conclusion of the predetermined period of time, the process 300 can return to the block 302 and thereafter execute as described previously. In various embodiments, the process 300 can continue until terminated by a user or system, or until suitable stop criteria is satisfied.

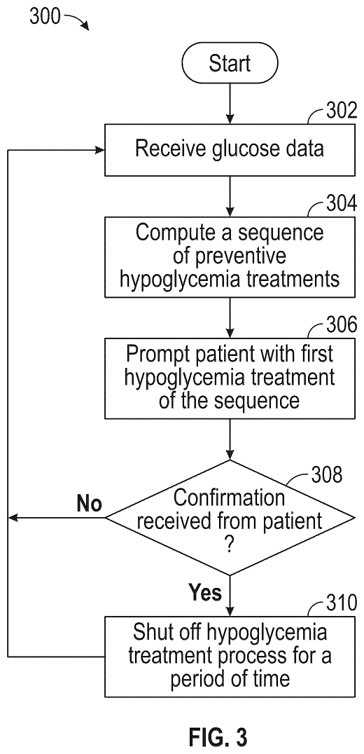

FIG. 4 is a line graph representing glucose concentration levels 406 and alternative treatments over time. Euglycemic range 407 is represented by an upper boundary 407a and a lower boundary 407b. Line 401 represents the glucose concentration levels 406 through a current time k. Branch-out lines 402a, 403a, and 404a represent predicted glucose concentration levels if preventive HTs 408 are taken at future times 402b, 403b, and 404b, respectively. Branch-out line 405 represents a reactive strategy in which no preventive CHO dosages are taken until after the glucose concentrations 406 cross the lower boundary 407b of the euglycemic range 407.

In the example of FIG. 4, the branch-out lines 403a, 404a, and 405 illustrate the predicted glucose concentration levels approaching or crossing the lower boundary 407b. In certain embodiments, according to the example algorithm described in Sections 1-5, the approach represented by the branch-out line 402a can be representative of the first HT in a computed sequence of HTs, as discussed relative to FIG. 3 and in Sections 1-5 above.

FIG. 5 illustrates an example of a penalty function D(q, k), as discussed above in Section 4.1.

FIG. 6 illustrates an example distribution of time below the targeted range (TBR) for five HT methods, as discussed above in Section 5.4

FIG. 7 illustrates hypoglycemic events for five HT methods, as discussed above in Section 5.4.

FIG. 8 illustrates example HTs for five HT methods, as discussed above in Section 5.4.

FIG. 9 illustrates a probability of selecting particular treatments for three HT methods, as discussed above in Section 5.4.

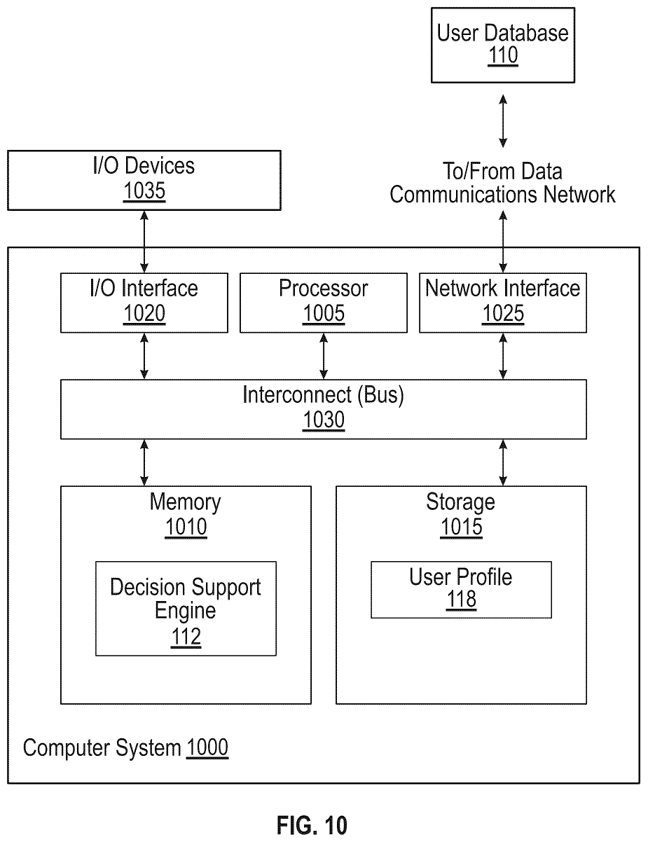

FIG. 10 is a block diagram depicting a computer system 1000 configured for facilitating preventive treatment of hypoglycemia, for example, according to certain embodiments disclosed herein. Although depicted as a single physical device, in embodiments, the computer system 1000 may be implemented using virtual device(s), and/or across a number of devices, such as in a cloud environment and/or via separate modules of portable or cloud devices. As illustrated, the computer system 1000 includes a processor 1005, a memory 1010, a storage 1015, a network interface 1025, and one or more I/O interfaces 1020. In the illustrated embodiment, the processor 1605 retrieves and executes programming instructions stored in the memory 1010, as well as stores and retrieves application data residing in the storage 1015. The processor 1005 is generally representative of a single CPU and/or GPU, multiple CPUs and/or GPUs, a single CPU and/or GPU having multiple processing cores, and the like.

The memory 1010 is generally included to be representative of a random access memory (RAM). The storage 1015 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, the I/O devices 1035 (such as keyboards, monitors, etc.) can be connected via the I/O interface(s) 1020. Further, via the network interface 1025, the computer system 1000 can be communicatively coupled with one or more other devices and components, such as the user database 110. In certain embodiments, the computer system 1000 is communicatively coupled with other devices via a network, which may include the Internet, local network(s), and the like. The network may include wired connections, wireless connections, or a combination of wired and wireless connections. As illustrated, the processor 1005, memory 1010, storage 1015, network interface(s) 1025, and the I/O interface(s) 1020 are communicatively coupled by one or more interconnects 1030. In certain embodiments, the computer system 1000 is representative of the display device 107 associated with the user. In certain embodiments, as discussed above, the display device 107 can include the user's laptop, computer, smartphone, and the like. In another embodiment, the computer system 1000 is a server executing in a cloud environment.

In the illustrated embodiment, the storage 1015 includes the user profile 118. The memory 1010 includes the decision support engine 112. The decision support engine 112 can be executed by the computer system 1000 to perform operations, for example, of the process 300 of FIG. 3.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### 7. References

The devices, structures, systems, computer program products, and methods of certain embodiments may utilize aspects disclosed in the following publications and which are hereby incorporated by reference herein in their entirety.
[1] P. E. Cryer, S. N. Davis, H. Shamoon, Hypoglycemia in Diabetes, Diabetes Care 26 (6) (2003) 1902-1912.
[2] G. Cappon, G. Acciaroli, M. Vettoretti, A. Facchinetti, G. Sparacino, Wearable Continuous Glucose Monitoring Sensors: A Revolution in Diabetes Treatment, Electronics (Switzerland) 6 (September 2017).
[3] K. Miller, N. Foster, R. Beck, R. Bergenstal, S. DuBose, L. DiMeglio, D. Maahs, W. Tamborlane, T1d exchange clinic network. current state of type 1 diabetes treatment in the u.s.: updated data from the t1d exchange clinic registry, Diabetes Care 38 (6) (2022) 971-8.
[4] R. Nimri, T. Battelino, L. L. et al., Insulin dose optimization using an automated artificial intelligence-based decision support system in youths with type 1 diabetes, Nat Med 26 (2020) 1380-1384.
[5] C. Fabris, B. Ozaslan, M. D. Breton, Continuous glucose monitors and activity trackers to inform insulin dosing in type 1 diabetes: the university of virginia contribution, Sensors 19 (24) (2019) 5386.
[6] M. D. Breton, S. D. Patek, D. Lv, E. Schertz, J. Robic, J. Pinnata, L. Kollar, C. Barnett, C. Wakeman, M. Oliveri, C. Fabris, D. Chernavvsky, B. P. Kovatchev, S. M. Anderson, Continuous glucose monitoring and insulin informed advisory system with automated titration and dosing of insulin reduces glucose variability in type 1 diabetes mellitus, Diabetes Technology & Therapeutics 20 (8) (2018) 531-540. doi: 10.1089/dia.2018.0079.
[7] A. Revert, R. Calm, J. Vehí, J. Bondia, Calculation of the best basal-bolus combination for postprandial glucose control in insulin pump therapy, IEEE Transactions on Biomedical Engineering 58 (2) (2010) 274-281.
[8] Q. Sun, M. V. Jankovic, S. G. Mougiakakou, Reinforcement learning based adaptive insulin advisor for individuals with type 1 diabetes patients under multiple daily injections therapy, in: 2019 41st Annual International Conference of the IEEE Engineering in Medicine and Biology Society (EMBC), IEEE, 2019, pp. 3609-3612.
[9] G. Noaro, G. Cappon, M. Vettoretti, G. Sparacino, S. Del Favero, A. Facchinetti, Machine-learning based model to improve insulin bolus calculation in type 1 diabetes therapy, IEEE Transactions on Biomedical Engineering 68 (1) (2020) 247-255.
[10] F. Prendin, S. Del Favero, M. Vettoretti, G. Sparacino, A. Facchinetti, Forecasting of glucose levels and hypoglycemic events: Head-to-head comparison of linear and nonlinear data-driven algorithms based on continuous glucose monitoring data only, Sensors 21 (5) (2021). doi: 10.3390/s21051647.
[11] C. C. Palerm, J. P. Willis, J. Desemone, B. W. Bequette, Hypoglycemia prediction and detection using optimal estimation, Diabetes technology & therapeutics 7 (1) (2005) 3-14.
[12] C. C. Palerm, B. W. Bequette, Hypoglycemia detection and prediction using continuous glucose monitoring—a study on hypoglycemic clamp data (2007).
[13] N. Camerlingo, M. Vettoretti, S. Del Favero, G. Cappon, G. Sparacino, A. Facchinetti, A real-time continuous glucose monitoring-based algorithm to trigger hypotreatments to prevent/mitigate hypoglycemic events, Diabetes Technology & Therapeutics 21 (11) (2019) 644-655. doi: 10.1089/dia.2019.0139.
[14] K. Turksoy, J. Kilkus, I. Hajizadeh, S. Samadi, J. Feng, M. Sevil, C. Lazaro, N. Frantz, E. Littlejohn, A. Cinar, Hypoglycemia detection and carbohydrate suggestion in an artificial pancreas, Journal of Diabetes Science and Technology 10 (6) (2016) 1236-1244. doi: 10.1177/1932296816658666.
[15] Y. Wu, X. Yao, G. Vespasiani, A. Nicolucci, Y. Dong, J. Kwong, L. Li, X. Sun, H. Tian, S. Li, et al., Mobile app-based interventions to support diabetes self-management: a systematic review of randomized controlled trials to identify functions associated with glycemic efficacy, JMIR mHealth and uHealth 5 (3) (2017) e6522.
[16] S. Veazie, K. Winchell, J. Gilbert, R. Paynter, I. Ivlev, K. B. Eden, K. Nussbaum, N. Weiskopf, J.-M. Guise, M. Helfand, Rapid evidence review of mobile applications for self-management of diabetes, Journal of general internal medicine 33 (7) (2018) 1167-1176.
[17] C. Liu, P. Avari, Y. Leal, M. Wos, K. Sivasithamparam, P. Georgiou, M. Reddy, J. M. Fernández-Real, C. Martin, M. Fernández-Balsells, N. Oliver, P. Herrero, A modular safety system for an insulin dose recommender: A feasibility study, Journal of Diabetes Science and Technology 14 (1) (2020) 87-96.
[18] J. Pavan, D. Salvagnin, A. Facchinetti, G. Sparacino, S. Del Favero, Incorporating sparse and quantized carbohydrates suggestions in model predictive control for artificial pancreas in type 1 diabetes, IEEE Transactions on Control Systems Technology (2022).
[19] A. D. Association, 2. classification and diagnosis of diabetes: Standards of medical care in diabetes—2019, Diabetes Care 42 (Supplement 1) (2019) S13-S28. doi: 10.2337/dc19-S002.
[20] M. Vettoretti, A. Facchinetti, G. Sparacino, C. Cobelli, Type-1 diabetes patient decision simulator for in silico testing safety and effectiveness of insulin treatments, IEEE Transactions on Biomedical Engineering 65 (6) (2018) 1281-1290. doi: 10.1109/TBME.2017.2746340.
[21] G. Cappon, G. Acciaroli, M. Vettoretti, A. Facchinetti, G. Sparacino, Wearable continuous glucose monitoring sensors: A revolution in diabetes treatment, Electronics 6 (3) (2017). doi: doi: http://dx.doi.org/10.3390/electronics6030065.
[22] B. P. Kovatchev, D. J. Cox, L. A. Gonder-Frederick, W. Clarke, Symmetrization of the blood glucose measurement scale and its applications, Diabetes Care 20 (11) (1997) 1655-1658. doi: 10.2337/diacare.20.11.1655.
[23] J. Yang, L. Li, Y. Shi, X. Xie, An arima model with adaptive orders for predicting blood glucose concentrations and hypoglycemia, IEEE Journal of Biomedical and Health Informatics 23 (3) (2019) 1251-1260. doi: 10.1109/JBHI.2018.2840690.
[24] C. Zecchin, A. Facchinetti, G. Sparacino, C. Cobelli, Jump neural network for online short-time prediction of blood glucose from continuous monitoring sensors and meal information, Computer methods and programs in biomedicine 113 (1) (2014) 144-152.
[25] J. I. Hidalgo, J. M. Colmenar, G. Kronberger, S. M. Winkler, O. Garnica, J. Lanchares, Data based prediction of blood glucose concentrations using evolutionary methods, Journal of medical systems 41 (9) (2017) 142.
[26] S. Oviedo, J. Vehí, R. Calm, J. Armengol, A review of personalized blood glucose prediction strategies for t1dm patients, International journal for numerical methods in biomedical engineering 33 (6) (2017) e2833.
[27] C. Zecchin, A. Facchinetti, G. Sparacino, C. Cobelli, How much is shortterm glucose prediction in type 1 diabetes improved by adding insulin delivery and meal content information to cgm data? a proof-of-concept study, Journal of diabetes science and technology 10 (5) (2016) 1149-1160.
[28] K. Li, J. Daniels, C. Liu, P. Herrero, P. Georgiou, Convolutional recurrent neural networks for glucose prediction, IEEE journal of biomedical and health informatics 24 (2) (2019) 603-613.
[29] A. L. McCall, Insulin therapy and hypoglycemia, Endocrinology and Metabolism Clinics of North America 41 (1) (2012) 57-87. doi: https://doi.org/10.1016/j.ecl.2012.03.001.
[+] A. Beneyto, A. Bertachi, J. Bondia, J. Vehi, A new blood glucose control scheme for unannounced exercise in type 1 diabetic subjects, IEEE Transactions on Control Systems Technology 28 (2) (2018) 593-600.
[31] C. Viñals, A. Beneyto, J.-F. Martín-SanJosé, C. Furió-Novejarque, A. Bertachi, J. Bondia, J. Vehi, I. Conget, M. Giménez, Artificial pancreas with carbohydrate suggestion performance for unannounced and announced exercise in type 1 diabetes, The Journal of Clinical Endocrinology & Metabolism 106 (1) (2021) 55-63.
[32] IBM, Ilog cplex optimization studio 12.9 user's manual (2019).
[33] S. D. Patek, L. Magni, E. Dassau, C. S. Hughes, C. Toffanin, G. D. et al., Modular closed-loop control of diabetes, IEEE Trans Biomed Eng 59 (11) (2012) 2986-2999.
[34] P. Davidson, H. Hebblewhite, R. Steed, B. Bode, Analysis of Guidelines for Basal-Bolus Insulin Dosing: Basal Insulin, Correction Factor, and Carbohydrate-to-Insulin Ratio, Endocr. Pract. 14 (9) (2008) 1095-1101.
[35] F. Simone, A. Facchinetti, G. Sparacino, G. Pillonetto, S. Del Favero, Linear model identification for personalized prediction and control in diabetes, IEEE Transactions on Biomedical Engineering 69 (2) (2021) 558-568.
[36] R. Visentin, E. Campos-Náñez, M. Schiavon, L. Dayu, M. Vettoretti, M. B. et al., The uva/padova type 1 diabetes simulator goes from single meal to single day, J Diabetes Sci Technol 12 (2018) 273-281.
[37] C. Dalla Man, F. Micheletto, D. Lv, M. Breton, B. Kovatchev, C. Cobelli, The uva/padova type 1 diabetes simulator: New features, J Diabetes Sci Technol 8 (2014) 26-34.
[38] R. Visentin, C. Dalla Man, Y. C. Kudva, A. Basu, C. Cobelli, Circadian variability of insulin sensitivity: Physiological input for in silico artificial pancreas, Diabetes Technol Ther. 17 (1) (2015). doi: https://doi.org/10.1089/dia.2014.0192.
[39] A. Facchinetti, S. D. Favero, G. Sparacino, C. Cobelli, Model of glucose sensor error components: identification and assessment for new dexcom g4 generation devices, Med Biol Eng Comput 53 (2015) 1259-69.
[40] A. S. Brazeau, H. Mircescu, K. Desjardins, C. Leroux, I. Strychar, K. M. Ekoé, R. R. Lhoret, Carbohydrate counting accuracy and blood glucose variability in adults with type 1 diabetes, Diabetes Res Clin Pract 99 (2013) (2012) 19-23.
[41] C. Roversi, M. Vettoretti, S. Del Favero, A. Facchinetti, G. a. Sparacino, Modeling carbohydrate counting error in type 1 diabetes management, Diabetes Technology & Therapeutics 22 (10) (2020) 749-759. doi: 10.1089/dia.2019.0502.
[42] M. J. Lenhard, G. D. Reeves, Continuous subcutaneous insulin infusion: a comprehensive review of insulin pump therapy, Archives of Internal Medicine 161 (19) (2001) 2293-2300.
[43] S. Schmidt, K. Nrgaard, Bolus calculators, J Diabetes Sci Technol 8 (5) (2014) 1035-1041.
[44] C. Ellingsen, E. Dassau, H. Zisser, B. Grosman, M. W. Percival, L. Jovanovi\grave{c}, F. J. Doyle III, Safety constraints in an artificial pancreatic β cell: an implementation of model predictive control with insulin on board, Journal of diabetes science and technology 3 (3) (2009) 536-544.
[45] T. Danne, R. Nimri, T. Battelino, R. M. Bergenstal, K. L. Close, J. H. DeVries, S. Garg, L. Heinemann, I. Hirsch, S. A. Amiel, R. Beck, E. Bosi, B. Buckingham, C. Cobelli, E. Dassau, F. J. Doyle, S. Heller, R. Hovorka, W. Jia, T. Jones, O. Kordonouri, B. Kovatchev, A. Kowalski, L. Laffel, D. Maahs, H. R. Murphy, K. Nørgaard, C. G. Parkin, E. Renard, B. Saboo, M. Scharf, W. V. Tamborlane, S. A. Weinzimer, M. Phillip, International Consensus on Use of Continuous Glucose Monitoring, Diabetes Care 40 (12) (2017) 1631-1640.
[46] D. M. Maahs, B. A. Buckingham, J. R. Castle, A. Cinar, E. R. Damiano, E. Dassau, J. H. DeVries, F. J. Doyle, S. C. Griffen, A. Haidar, L. Heinemann, R. Hovorka, T. W. Jones, C. Kollman, B. Kovatchev, B. L. Levy, R. Nimri, D. N. O'Neal, M. Philip, E. Renard, S. J. Russell, S. A. Weinzimer, H. Zisser, J. W. Lum, Outcome Measures for Artificial Pancreas Clinical Trials: A Consensus Report, Diabetes Care 39 (7) (2016) 1175-1179. doi: 10.2337/dc15-2716.
[47] B. Grosman, E. Dassau, H. C. Zisser, L. Jovanovi\grave{c}, F. J. Doyle III, Zone model predictive control: a strategy to minimize hyper- and hypoglycemic events, Journal of diabetes science and technology 4 (4) (2010) 961-975.
[48] C. Toffanin, M. Messori, F. Di Palma, G. De Nicolao, C. Cobelli, L. Magni, Artificial pancreas: model predictive control design from clinical experience, J. Diabetes Sci. Technol. 7 (6) (2013) 1470-1483.

1. A system for preventively treating hypoglycemia, comprising:
a continuous glucose monitoring (CGM) sensor system configured to generate measurements associated with a current glucose level of a patient;
one or more memories comprising executable instructions; and
one or more processors in data communication with the one or more memories and configured to execute the executable instructions to:
receive, from the CGM sensor system, a first one or more measurements associated with the current glucose level of the patient;
compute a first sequence of preventive hypoglycemia treatments over a first future time period based on the first one or more measurements and a prediction of glucose control to be produced by the first sequence; and
prompt the patient with a first preventive hypoglycemia treatment in the first sequence of preventive hypoglycemia treatments.

2. The system of claim 1, wherein the computation of the first sequence comprises determining that the first sequence minimizes a cost function, the cost function penalizing an increase in number of hypoglycemia treatments during the first future time period.

3. The system of claim 2, wherein the first sequence is computed via a constrained integer program based on the minimized cost function.

4. The system of claim 2, wherein the cost function excludes consideration of dosage amounts.

5. The system of claim 2, wherein the cost function further penalizes an increase in distance between a plurality of predicted glucose levels and a euglycemic setting.

6. The system of claim 1, wherein the computation of the first sequence comprises enforcing a minimal distance in time between consecutive hypoglycemia treatments of the first sequence of preventive hypoglycemia treatments by imposing one or more linear inequality constraints on the first sequence.

7. The system of claim 1, wherein each preventive dosage in the first sequence is selected from a plurality of predetermined dosage amounts.

8. The system of claim 1, wherein the first sequence of preventive hypoglycemia treatments is computed by a preventive hypoglycemia treatment process that is executed by the one or more processors at a regular interval, and the one or more processors are further configured to execute the executable instructions to:
receive a confirmation that the first preventive hypoglycemia treatment has been delivered to the patient; and
responsive to the receipt of the confirmation, shut off the preventive hypoglycemia treatment process for a predetermined time period.

9. The system of claim 8, wherein the one or more processors are further configured to execute the executable instructions to:
receive, from the CGM sensor system, a second one or more measurements associated with the current glucose level of the patient;
after the predetermined time period, compute a second sequence of preventive hypoglycemia treatments over a second future time period based on the second one or more measurements and a prediction of glucose control to be produced by the second sequence, wherein the second sequence of preventive hypoglycemia treatments modifies the first sequence of preventive hypoglycemia treatments; and
prompt the patient with a first preventive hypoglycemia treatment in the second sequence of preventive hypoglycemia treatments.

10. The system of claim 1, wherein the first one or more measurements fail to satisfy a threshold associated with hypoglycemia.

11. The system of claim 1, wherein the first sequence of preventive hypoglycemia treatments each comprise a carbohydrate dosage.

12. A method of preventively treating hypoglycemia, the method comprising:
at a user device, receiving, from a continuous glucose monitoring (CGM) sensor system, a first one or more measurements associated with a current glucose level of a patient;
computing, at the user device, a first sequence of preventive hypoglycemia treatments over a first future time period based on the first one or more measurements and a prediction of glucose control to be produced by the first sequence; and
at the user device, prompting the patient with a first preventive hypoglycemia treatment in the first sequence of preventive hypoglycemia treatments.

13. The method of claim 12, wherein the computing the first sequence comprises determining that the first sequence minimizes a cost function, the cost function penalizing an increase in number of hypoglycemia treatments during the first future time period.

14. The method of claim 13, wherein the first sequence is computed via a constrained integer program based on the minimized cost function.

15. The method of claim 13, wherein the cost function excludes consideration of dosage amounts.

16. The method of claim 12, wherein the computing the first sequence comprises enforcing a minimal distance in time between consecutive hypoglycemia treatments of the first sequence of preventive hypoglycemia treatments by imposing one or more linear inequality constraints on the first sequence.

17. The method of claim 12, wherein each preventive dosage in the first sequence is selected from a plurality of predetermined dosage amounts.

18. The method of claim 12, wherein the first sequence of preventive hypoglycemia treatments is computed by a preventive hypoglycemia treatment process that is executed by the user device at a regular interval, and the method further comprises:
receiving, at the user device, a confirmation that the first preventive hypoglycemia treatment has been delivered to the patient; and
responsive to the receipt of the confirmation, shutting off the preventive hypoglycemia treatment process for a predetermined time period.

19. The method of claim 18, further comprising:
at the user device, receiving, from the CGM sensor system, a second one or more measurements associated with the current glucose level of the patient;
after the predetermined time period, computing a second sequence of preventive hypoglycemia treatments over a second future time period based on the second one or more measurements and a prediction of glucose control to be produced by the second sequence, wherein the second sequence of preventive hypoglycemia treatments modifies the first sequence of preventive hypoglycemia treatments; and
prompting the patient with a first preventive hypoglycemia treatment in the second sequence of preventive hypoglycemia treatments.

20. A computer-program product comprising a non-transitory computer-usable medium having computer-readable program code embodied therein, the computer-readable program code adapted to be executed to implement a method comprising:
receiving, from a continuous glucose monitoring (CGM) sensor system, a first one or more measurements associated with a current glucose level of a patient;
computing a first sequence of preventive hypoglycemia treatments over a first future time period based on the first one or more measurements and a prediction of glucose control to be produced by the first sequence; and
prompting the patient with a first preventive hypoglycemia treatment in the first sequence of preventive hypoglycemia treatments.
